(19) United States
(12) Patent Application Publication
(10) Pub. No.: US 2023/0339988 A1
(43) Pub. Date: Oct. 26, 2023

(54) Metal Complexes Of Macrocycles And/Or Isoprenoids And/Or Linear Tetrapyrroles By Mechanochemistry (Grinding Or Milling), Preparation Method Thereof, Sunscreen/Concealer/UV Absorber Thereof, Self-Assembled Coating Material Thereof, Superamphiphilic Material Or Surfaces Thereof, Hair Dyeing Thereof And Other Uses Thereof

(71) Applicant: Liliana Patricia Ramirez Rios, The Hague (NL)

(72) Inventor: Liliana Patricia Ramirez Rios, The Hague (NL)

(21) Appl. No.: 17/787,435

(22) PCT Filed: Dec. 16, 2020
(86) PCT No.: PCT/EP2020/000213

§ 371 Date: Jun. 20, 2022

(30) Foreign Application Priority Data
Dec. 18, 2019 (EP) 19075019.0
Jun. 10, 2020 (EP) 20075007.3

(51) Int. Cl. C07F 7/28; C07F 19/00; A61K 8/29; A61Q 5/10; A61Q 17/04

(52) U.S. Cl. CPC C07F 7/28; C07F 19/00; A61K 8/29; A61Q 5/10; A61Q 17/04; A61K 2800/43

(57) ABSTRACT

Metal complexes of macrocycles and/or isoprenoids and/or linear tetrapyrroles by mechanochemistry (grinding or milling), preparation method thereof, sunscreen/concealer/UV absorber thereof, self-assembled coating material thereof, superamphiphilic material or surfaces thereof, hair dyeing thereof and other uses thereof. Converting biomass, including products from its own process-line wastes, into high value products such as biofuel, bioplastics and biochemicals in an attempt to replace oil consumption has become nowadays a challenge for innovation. In one embodiment of the present invention, a novel product obtained from the solvent-free mechanochemical reaction (grinding and milling) of biomass and a metal alkoxide is produced. In one embodiment of the present invention, Spirulina biomass, comprising macrocycles (chlorophyll) and isoprenoids (P-carotene) and linear tetrapyrroles (phycobilins attached to proteins), is ground or milled together with a metal alkoxide to produce a stable colored material.

### DESCRIPTION OF THE INVENTION

In nature, endogenous processes do not need extreme conditions, hazardous reagents or solvents to take place. For instance, the light harvesting systems of photosynthetic organisms consist of numerous macrocycles (e.g. chlorophylls), isoprenoids (e.g. carotenoids) and linear tetrapyrroles (e.g. phycobilins or phycocyanin) dyes/compounds which are bound by several interactions to proteins (e.g. phycobiliproteins) and/or metals (e.g. Mg in chlorophyll). All these dyes involved in this natural process have a specific function and interact with the surrounding molecules to accomplish their purpose. Macrocycles and bilanes—the fundamental linear tetrapyrrole system—act as light harvesting antennae and isoprenoids act as photoprotecting pigments. Sometimes the terms bilanes and bilins are used as synonyms of linear tetrapyrroles compounds. In the present invention, I opt for the term linear tetrapyrroles as in IUPAC recommendations. By this way, linear tetrapyrroles comprises also bilins and bilanes with or without oxygen substituents and/or with saturated or unsaturated nitrogens.

The present invention relates to a method to synthesize with high yield a colored metal complex of macrocycles and/or isoprenoids and/or linear tetrapyrroles by mechanochemistry under solvent-free conditions and covers new complexes obtained therefrom. In an embodiment, only the reactants, a metal alkoxide and a macrocycle and/or an isoprenoid/terpenoid and/or a linear tetrapyrrole, are present and react at different molar ratios to form a metal complex in the form of a homogeneous colored material (e.g. powder) with high yield. If the macrocycle phthalocyanine is used, the color of the final metal complex dye depends on several factors such as the metal, the type of alkoxide used, the stoichiometric molar ratio of both reactants and the additives used. If titanium alkoxide and no additive is used, a blue colored titanium macrocycle complex in powder form is produced. The production process of the metal complex in powder form is characterized by facility of scale up. The entire product is ready to be used in several applications. If other macrocycle compounds (e.g. porphyrin and calixarenes) and/or isoprenoids and/or linear tetrapyrroles are used instead of phthalocyanine, other differently colored complexes are obtained using mechanochemistry. In addition, the product comprising metal complexes of macrocycles and/or isoprenoids and/or linear tetrapyrroles is characterized by green chemistry manufacture. Besides, these metal complexes of macrocycles and/or isoprenoids and/or linear tetrapyrroles in several formulations are characterized by improved stability under storage conditions and improved sun protection against UV rays for human skin and human hair and enhanced amphiphilic properties and/or suprahydrophobocity and/or suprahydrophilicity. The product is ready to be used alone or in combination with other additives (e.g. titanium oxide) or active ingredients in different formulations. The invention also covers the use of these metal macrocycle/isoprenoid/linear tetrapyrrole complexes in food, cosmetics, the pharmaceutical field and in the creativity field, as sunscreen, skin and hair concealer or foundation in powder form or cream, keratinous dye, textile dye, food dye, dye emulsions, miniemulsions, polymer colloids and catalysts and coatings.

The production process of these novel metal-complex dyes is also characterized by rapid production of colored compounds without using solvents and, thus, ecofriendliness. The process of the production of metal macrocycle and/or isoprenoid/terpenoid and/or linear tetrapyrrole complexes is characterized by the non-formation of toxic by-products and high yield.

In addition, the present invention comprises a hair dyeing molecule or formulation and procedure combining excellent dyeing properties with reduced risk of development of cancer or allergies, while being ecological and of natural origin.

The present invention provides a process for the synthesis of metal complexes of macrocycles (e.g. metal or metal-free phthalocyanines, metal or metal-free porphyrins and calixarenes) and/or isoprenoids (e.g. β-carotene, squalene) and/or linear tetrapyrrole (e.g. bilirubin) by mechanochemistry and a food, a cosmetic, a pharmaceutical or a paint formulation thereof and other uses thereof. These formulations are colored and exhibit an excellent stability in the form of powder or colloids. In particular, the titanium phthalocyanine complexes are greenish-blue colored; the titanium meso-tetraphenylporphine complexes are reddish-purple and the titanium calixarene complexes are yellow, among others. The color palette is very wide and varies with changes in the reactants and the conditions of the mechanochemical reaction (grinding or milling) and the final formulation. Small changes in the formulation can produce a marked difference in color which may be blue, violet, green, yellow, red, brown or black. The sunscreen formulations with concealer effect using the metal complex dyes of the present invention are stable and do not fade. The present invention provides also a temporal and semipermanent hair dye and a hair dyeing process with excellent dyeing properties.

The process of the production of the metal macrocycle/isoprenoid/linear tetrapyrrole complexes of the present invention provides a new method of functionalization of surfaces/interfaces by mechanochemistry (grinding and milling) and at the same time enhances the amphiphilic properties of the initial counterparts.

### BACKGROUND OF THE INVENTION

Macrocycle Ligands

According to IUPAC,^{1,2} macrocycle is defined as a cyclic macromolecule or a macromolecular cyclic portion of a macromolecule. Note 1: in the literature, the term macrocycle is sometimes used for molecules of low molecular mass that would not be considered ‘macromolecules’ as specified in the definition given in the IUPAC book. Note 2: a cyclic macromolecule has no end groups but may nevertheless be regarded as a chain. A cyclic macromolecule is a macromolecule in which the termini of the backbone are connected to form a ring.

Macrocyclic ligands³ are cyclic molecules generally consisting of organic frames into which heteroatoms, capable of binding to substrates, have been interspersed.

Macrocycles can be naturally occurring compounds, such as porphyrins, corrins, corroles, chlorins, corphin, bacteriochlorins, chlorophylls, sapphyrin and synthetic compounds, such as phthalocyanines, cyclodextrins, calixarenes. Many of these macrocycles in the nature are always complexed with metals such as chlorophylls (magnesium-containing chlorins) or haem (iron-containing porphyrin). Some synthetic macrocycles such as phthalocyanine are more easily produced and commercialized in the metal complex such as copper phthalocyanine than in the metal free phthalocyanine. Supramolecular macrocyclic structures such as calixarenes can trap, inside or outside their structure, small molecules such as metals.

Since macrocycle ligands can interact with metals through different kinds of bonds (non-covalent or covalent), the term complexes is preferred to supramolecular complexes, where only the non-covalent bond is assumed.

Macrocyclic Effect

The macrocyclic effect is a chelate effect further enhanced by the cyclic structure of the ligand. The macrocyclic effect is a strong affinity of poly-dentate ligands which are covalently constrained to their cyclic form for metal ions. Macrocyclic compounds are predestined for binding.

In coordination chemistry, A. Werner developed the oldest classical type of synthesis of coordination compounds involving the reaction of metal salts with a ligand in a liquid medium. Bearing this in mind, it is not surprising that most of the new supramolecular complexes use metal salts as a metal source and a solvent.

In the present patent the term metal complexes is generally used but it does not mean the metal ions are contained in the centrum of the complexes. The metal ion(s) may be in an axial position or in another position such as in the case of coordination complexes or compounds.

Macrocycles and/or Isoprenoids and/or Linear Tetrapyrroles

Tetrapyrroles and their Relatives

According to IUPAC,⁴ tetrapyrroles contain four five-membered heterocyclic (pyrrole) rings generally linked together by single-atom bridges between the alpha positions of the five-membered pyrrole rings, being the alpha positions of the pyrrole ring those adjacent to the nitrogen atom. The commons arrangements of the four rings are macrocyclic (e.g. porphyrins, or phthalocyanines) and linear (e.g. bilanes).

Bilins are considered more similar to chlorophylls, as the pseudo-cyclic structure is reminiscent of macrocycles tetrapyrroles.

Some tetrapyrrole macrocycles such as chlorophyll and heme (or haem) present a terpenoid chain in their structure. Both macrocycles and isoprenoids are found in the same structure in nature.

Porphyrins and Metallo-Porphyrins (Metalloporphyrinoids) and Linear Tetrapyrroles

Porphyrin and metallo-porphyrins are very common in nature, e.g. haem and siroHaem (Fe²⁺ as complexed metal ion), chlorophyll and bacteriochlorophyll (Mg²⁺ as complexed metal ion or other metals in synthetic chlorophyll), cobalamin (Co²⁺ as complexed metal ion) and coenzyme F₄₃₀ (Ni²⁺ as complexed metal ion). Chrichton (2019) describes the relationship between tetrapyrrole biosynthetic pathways⁵ as being uroporphyrinogen Ill, the cyclic tetrapyrrole that is common to all tetrapyrrole pathways. In turn, uroporphyrinogen Ill is formed from 5-aminolevunic acid in several enzymatic steps via the intermediates porphobilinogen and pre-uroporphyrinogen (1-hydroxymethylbilane, a linear tetrapyrrole) in the haem biosynthesis.

Fullerenes and Related Materials

Porphyrins and phthalocyanines are fully-conjugated planar systems and, similarly, fullerenes (e.g pristine or empty, endohedral or truncated fullerenes), being curved ligands with spherical π-conjugation, are used as macrocyclic ligands. Fullerenes are capable of inclusion complex formation, since they can host ions and molecules inside or outside their cage, and they play the guest roll of being buried inside another cage or capsule. Several chemical reactions can be performed from fullerene (e.g. C₆₀) but to date the mechanochemical reaction with a metal alkoxide has not been reported.

Isoprenoids (Sometimes Called Terpenoids)

According to IUPAC,¹ isoprenoids are compounds formally derivated from isoprene(2-methylbuta-1,3-diene). The skeleton of which can generally be discerned in the repeated occurrence in the molecule. The skeleton of isoprenoids may differ from strict additivity of isoprene units by loss or shift of a fragment, commonly a methyl group. The class includes both hydrocarbons and oxygenated derivatives.

According to IUPAC,¹ terpenoids are natural products, and related compounds also formally derived from isoprene units and their skeleton may differ from strict additivity of isoprene units by the loss or shift of a fragment such as in the case of isoprenoids. They contain oxygen in various functional groups. This class is subdivided according to the number of carbon atoms in the same manner as are terpenes. Thus, a terpenoid is an isoprenoid of natural origin. Sometimes, the terms are considered synonyms. In the present description the term isoprenoids will be used including the isoprenoids of natural origin i.e. terpenoids.

The chlorophyll and heme have isoprenoid side chains in their structures.

In nature, the metal complexation is not an isolated process. In plants, chlorophylls, already complexed with magnesium, use isoprenoids (e.g. carotenoids) and linear tetrapyrroles, the so-called photosynthetic pigments, to synthetize organic matter with the help of light within the photosynthetic machinery, whereas other enzymes and proteins are also involved in this process.

Carotenoids

According to IUPAC,¹ carotenoids are tetraterpenoids (C₄₀), formally derived from the acyclic parent, ψ, ψ-carotene by hydrogenation, dehydrogenation, cyclization, oxidation, or a combination of these processes. This class includes carotenes, xanthophylls and certain compounds that arise from the rearrangement of the skeleton of ψ, ψ-carotene or by loss of part of this structure.

Carotenoids as well as porphyrinoids are pigments widely distributed in nature and their physical, chemical and biological properties are well known. The variety of colors of more than a thousand carotenoids is always directly linked to their structure. Carotenoids and chlorophylls work always one in conjunction with the another. Carotenoids are so-called accessory pigments in phothosynthesis.

Carotenoids linked to porphyrin or chlorophyll derivatives serve as molecular dyads for studies of photosynthesis and electron transfer. In addition, carotenoporphyrins are dyads which absorb the light in wavelengths different to those of their original counterparts.

In the present invention, isoprenoids (e.g. carotenoids) and macrocycles (e.g. chlorophylls) are complexed with metals to resemble natural photosynthesis.

Linear Tetrapyrroles and their Derivatives

Linear tetrapyrroles, such as bilanes are natural pigments formed by the metabolic product of certain porphyrins.

Phycobilins belong to the linear tetrapyrroles such as pre-uroporphyrinogen and are a linear arrangement of tetrapyrroles that represent the photosynthetic accessory pigments of several cyanobacteria and some eukaryotic algae. These pigments are covalent bound to phycobiliproteins.

In nature, phototropic organisms produce a diversity of pigments that have a biochemical function. Photosynthetic pigments are chlorophyll (e.g. a, b and c) and their accessory pigments such as carotenoids or phycobilins which are associated with the photosynthesis system. Their functions can be classified according to roles that are related to light harvesting (e.g. chlorophyll, fucoxanthin and phycobilins) or photoprotection/sunscreen (e.g. carotenoids, scytonemin).

Natural Resources

Natural products are a rich source of macrocycles and isoprenoids and linear tetrapyrroles in the same species such as plant, microbial sources and marine sources. Essential oils of plant origen are mainly terpenoid materials which are formed from two isoprene units joined together in a head-tail fashion. In fact the pioneer work on mycrocycles was reported on terpenoid macrocycles.⁶

In addition to the curcuminoids present in Curcuma L. and Zengiber officinali and related families such as Zingiberaceae, more than 250 mono-, sesqui-, di-, and triterpenoids have been identified.⁷ Bisabolane-type sesquiterpenes, germacrene isomeric sesquiterpenes and monoterpenes and other terpenoids are present. Some of these terpenoids are also macrocycles such as S-caryophyllene, germacrone, and guaiane.

Cannabinoids

Spirulina

Spirulina is the biomass of the cyanobacteria of Arthrospira species. In particular, Spirulina platensis powder is the powder obtained after washing, to remove adherent salts, and rapidly drying of the harvested biomass, Spirulina platensis. This powder is a source of proteins/phycobiliproteins (e.g. phycocyanin), chlorophylls, carotenoids, polyunsaturated fatty acids and vitamins, which are used as food additive.

Phycocyanin is a blue color that can be extracted from the spirulina biomass by several extraction techniques used in the extraction of biomolecules, such as soxhlet, solid-liquid extraction, liquid-liquid extraction, hydrodistillation, ultrasound- and microwave-assisted extraction, supercritical fluid extraction.

Both spirulina (blue and green powder or extract) and phycocyanin (blue pigment) can be obtained commercially as a superfood or food colorant.

Metal Alkoxide

Metal alkoxides⁸ are very versatile since they exhibit great differences in physical properties. They can be solid, non-distillable or distillable compounds or sublimable solids or liquids with different volatility character. The color of the metal alkoxide is originating from the metal ions.

Metal Macrocycles

Approaches for Synthesis of Metal Phthalocyanines/Porphyrins

According to W. A. Buchler,⁹ metal phthalocyanines can be synthesized from free phthalocyanines:

$${H}_{2} ( P ) + {MX}_{2} \begin{matrix}{\to }^{1 a} \\ {←}_{1 b}\end{matrix} M ( P ) + 2 HX$$

where in most cases H₂P is the porphyrin-free acid that reacts with a metal salt MX₂ producing the metallo-porphyrin M(P) and the corresponding acid HX. The process 1a is the metalation and the reverse 1b is the demetalation. According to Buchler, the metal alkoxide route is always in the presence of solvent such as pyridine and metallic magnesium powder to stabilize the porphyrin formed and for alkali metals and alkaline earth metals, since this kind of alkali or alkaline earth metal porphyrins are very sensitive to acid. W. Herbst and K. Hunger¹⁰ (2018) in Industrial Organic Pigments described methods to produce metal phthalocyanines

Typically, the syntheses of metal complexes of macrocycles such as porphyrin/phthalocyanines are by conventional solution methods. In solution-based or conventional methods of synthesis of phthalocyanines, a phthalic anhydride or a derivative thereof is heated under reflux along with a metal source, urea or a derivative thereof and a catalyst at a temperature of 180° C. to 300° C. either in the presence or absence of an organic solvent to allow them to react. These final pigments are coarse (crude metal phthalocyane) and require a reduction of the particle size in order to favorece further applications such as paintings and coatings. Therefore, the size is generally reduced after the synthesis of the crude pigments by grinding or milling. This is the so-called pigmentation step.

Grinding for conditioning organic pigments is a common technique to enhance the properties of pigments. Crude phthalocyanines are converted from its crude state to its pigmentary form by grinding or milling, commonly in combination with grinding media and other inorganic/organic pigments.

U.S. Pat. No. 5,318,623¹¹ disclosed a process for producing metal phthalocyanine pigments of a fine particle size, whereby the metal phthalocyanine pigment is synthesized by conventional methods (high temperatures and solvents) using metal salts and allowing the mixture to react, while simultaneously applying a mechanical grinding force with or without grinding aid (i.e. pigmentation step). In these reactions, solvent is used in huge quantities to let the reactants react or boil under reflux conditions at higher temperatures. As written in the examples 1 and 9, the purification processes of the crude product are not avoided and necessary in order to extract the large amounts of solvents used.

The metal source by the conventional solution methods are commonly metal salts, metal halides, metal alkoxides.

JP1994293769¹² disclosed a conventional solution method for producing oxytitanium phthalocyanine by the reaction of phthalonitrile and titanium alkoxide as a metal source in presence of urea, amide or thioamide compound or ammonium whereby the use of solvents was recommended as being industrially advantageous.

JP2003183534 disclosed a similar method as described by the previous patent '769 but dihydric alcohol was used as a more environment-friendly solvent.

The present invention of metal complexes of macrocycles deals with the grinding or milling of at least one macrocycle and at least one metal alkoxide together in this way, a mechanochemical reaction of both reactants, which are prone to react, promote the synthesis of clean metal complexes with small particle size. The reaction is favored by both the mechanochemical reaction (by grinding or by milling) and the macrocyclic effect. The solventless condition of the present invention in combination with grinding or milling of two compounds enhances the process of spontaneous self-assembly and the formation of very stable colored material. No exhausting work-up steps are needed since no salts are added as reactants.

In K. Ralphs et al.¹³ (2017), the synthesis of porphyrin metal complexes was made by mechanochemical synthesis of porphyrins using solid metal salts such as metal acetate x-hydrated salts of zinc, nickel, copper and iron.

When metals such as hydrozincite [Zn₅(CO₃)₂(OH)₆] or tetrakis(acetonitrile)copper(I)hexafluorophosphate {[Cu(CH₃CN)₄]PF₆} were used, the mechanochemical syntheses were unsuccessful both solventless or upon addition of small amounts of solvents. They conclude that the acetic acid that is eliminated in the reaction promote the metallation. However, metallations with other metals such as Au, Mg, Ag, Pt, Li, Mn, and Co were also unsuccessful despite using acetate salts and changing variables in ball milling such as milling speed, milling duration, and adding small amounts of solvent. Successful metallation was achieved only in the case of Zn, Ni, Cu, and Fe using solid metal salts such as acetate salts of the corresponding metals.

A. O. Atoyebi and C. Bruckner¹⁴ (2019) extended the work of Ralphs et al. (2017) on mechanochemical insertion of metal ions onto porphyrins to more metal sources and to other porphyrins. The metal sources were all salts such as acetates, perchlorates, chloride, bromides, iodides, sulfides and metal powder (e.g. Zn) and metal oxides. Besides, they used hydrozincite [Zn5(CO3)2(OH)6], Florisil and VO(acac)2.

Both abovementioned approaches are simple ball milling to intensively grind together solid reagents to induce a reaction as highlighted by Atoyebi and Bruckner. Both the porphyrins and the metal source (e.g. metal salts) are solids and, thus, the described mechanochemical process is rather a cocrystalisation process.

In the present invention, metal complexes of macrocycle compounds are formed by milling at least one macrocyclic ligand and at least one metal alkoxide together via a one-mortar self-assembly of metal alkoxide and a macrocyclic ligand by mechanochemical reaction. A metal alkoxide is used as a metal source, and only a macrocyclic ligand and a metal alkoxide are used under solventless conditions or in the presence of a minimal amount of solvent. Given the fact that both macrocycles and metal alkoxide may have different solubilities, depending of the branching of the alkyl chain or the substitution in the macrocycle, an appropriate selection of the reactants and the additives in the mechanochemical reaction would be advantageous.

One of the objects of the present invention of the complexation of macrocycles with a metal (e.g. titanium or zinc or both) is the enhancement of the properties of both components, such as the UV shielding, antimicrobial, anti-dandruff, anti-acne effect. Another purpose of the present invention is to produce a coloring matter such as blue, red, yellow, violet, green, brown and other with healthy properties using less harmful reactants.

Phthalocyanines and Metal Phthalocyanines

Phthalocyanines are structurally related to porphyrins. Formally, phthalocyanines are a tetrabenzotetraazaporphyrine (H₂P), which is the porphyrin analogue and the condensation product of four isoindole units (Ullmann's Encyclopedia of industrial chemistry). They are also similar to naturally occurring porphyrins such as hemoglobin, chlorophyll a, vitamin B₁₂. Phthalocyanines themselves do not occur in nature. Compounds with naphthalene or anthracene rings in place of the benzene nucleus also belong to the phthalocyanine family.

JP1996311365¹⁵ disclosed a modified chlorogallium phthalocyanine crystal by treating the chlorogallium phthalocyanine crystal with a solution of an alkali metal alkoxide.

The so-called solution of a alkali metal alkoxide was made by reacting metal amide or metal hydride or metal hydroxide with an alcohol. According to M. Horn,⁸ only the dialkylamides of some metal such as vanadium, chromium or niobium react with alcohols to give the corresponding metal alkoxide. In the case of sodium amide, alcohol is used usually for neutralizing sodium amide.

If metal hydroxide (e.g. sodium hydroxide) is used to react with an alcohol, the water liberated in the reaction must be removed to favor the equilibrium for the production of the metal alkoxide. Otherwise the water liberated during the reaction will favor the hydrolysis of the sodium alkoxide recently produced. Thus, the alcohol would only dissolve the sodium alkoxide.

As explained by Buchler,⁹ the metal alkoxide route with alkali metal such as lithium, sodium or potassium is always in the presence of a solvent or basic media in order to stabilize the metal porphyrin that could be formed if a free-phthalocyanine is used. Since chlorogallium phthalocyanine and a basic solution were used in '365, they followed the recommendations.

Phthalocyanine blues are the copper phthalocyanine. Metal-free phthalocyanine is also blue but is less used, since it is less brilliant in shade. Phthalocyanine greens are produced by halogenation of copper phthalocyanines.

Metal phthalocyanines possess a large delocalized n electron system, which exhibits useful electronic and photochemical properties.

Copper phthalocyanine (CI pigment blue 15) is perhaps the most important metal complex pigment since they have outstanding fastness properties.

In one embodiment of the present invention, a metal macrocycle complex is formed by the organic and aqueous solvent-free reaction of macrocycles with a metal alkoxide at room temperature by mechanochemistry (grinding or milling). The metal precursor is a metal alkoxide, and the macrocycle chelating agent is preferably phthalocyanine or a metal phthalocyanine (e.g. copper phthalocyanine). Water, if present, is from the atmosphere or generated during the reaction. Water or the parental alcohol from the alkoxide is supposed to be expelled during the reaction, but the metal macrocycle complex obtained in this invention is a homogeneously colored powder without precipitation or formation of two phases. The process is, presumably, a supramolecular arrangement of the molecules, where either water or the parental alcohol is included in the supramolecular structure.

The reaction is at room temperature, and only hand or mechanic maceration is required. Optionally, a moderate increase of the temperature is needed to reach the temperatures in a mortar and pestle which are commonly generating in ball milling. Solvents can generate artifacts that hinder or promote the interaction of the reactants and hinder the synthesis of the desired product. The present invention of the synthesis of metal macrocyclic complexes from the mechanochemical reaction of a metal alkoxide and a macrocycle and/or an isoprenoid and a linear tetrapyrrole is characterized by a solvent-free grinding or by solvent-free milling.

Hence, no artifacts are created by the use of solvents within the reaction that alter the interaction of the reactants. The present invention is also characterized by an economically attractive and straightforward implementation of large scale production. The most remarkable advantage of the present invention is that it fulfils all requirements to be a green synthesis.

Calixarenes

M. Czackler et al. (2014)¹⁶ prepared calixarene derivatives of titanium and zirconium alkoxides by using Schlenk techniques under inert gas atmosphere. To prepare titanium calixarene derivatives, titanium butoxide (1:1 adduct with butanol) was added to a suspension of calixarene (1:1 adduct in toluene) in butanol. The red metal calixarene bearing toluene was obtained after heating and recrystallization in toluene after two weeks. A similar procedure was used with zirconium butoxide with and without acetic acid or water, and clear crystals were obtained after 4 to 126 days depending on the initials conditions.

In the present invention, metal alkoxide and macrocycle calixarene are ground (or milled) in a mortar by using a pestle without using any kind of additives or solvents at several metal alkoxide macrocycle molar ratios. If titanium alkoxide and calixarene are used, the final powdered complex is yellow with unique superamphilicity properties never reported until now (See example 3). Mechanochemical solvent-free reaction of a macrocycle with a metal alkoxide using a mortar and a pestle, is by this way, a beneficial alternative to the conventional synthetic complexation in solution or by stirring, avoiding the use of sophisticated and troublesome techniques to handle the reactants. Even a mortar and a pestle without any kind of sealing can be used to perform a metal organic reaction. Without being linked to any theory, I speculate that the immediate interaction of the reactants by tribo mechanochemical energy promotes the confinement effect and protects the reaction from external factors such as moisture and atmosphere conditions.

Calixarenes are generally functionalized with aza structures, e.g. azacalixarenes to accept hosts such as metal (cations). Another approach to favor the complexation of calixarene with metals is the use of calix[4]pyrroles.

In the book calixarenes and beyond,¹⁷ P. Neri et al. reviewed the basic chemistry of the calixarene family including the synthesis or preparative procedures and functionalization methods. They reaffirmed that heterocycles having nitrogen in their structure are convenient for metal-driven self-assembly into macrocyclic structures such as metallacalixarenes. For the formation of metallated crypthophanes, a ruthenium metal complex, [Cp*Ru(CH₃CN)]⁺[SbF₆]⁻, was used.

In the present invention, in order to produce a metal complex of calixarene, only a simple host consisting of a calixarene without having nitrogen, sulfur or aza groups in its structure and a metal alkoxide are ground together in a mortar using a pestle.

Mechanochemistry

According to IUPAC,¹ a mechanochemical reaction is a chemical reaction that is induced by direct absorption of mechanical energy. Note: shearing, stretching, and grinding are typical methods for the mechanochemical generation of reactive sites, usually macroradicals, in polymer chains that undergo mechanochemical reactions.

The mechanochemical energy such as those caused by shearing, stretching, grinding and milling is the crucial activator of the mechanochemical reaction. Mechanochemistry is the fourth way of chemical activation after thermochemistry (tradicional thermal reactions), electrochemistry and photochemistry. Since term mechanochemistry was proposed by Ostwald in 1919, i.e. one hundred years ago, however, their use as a tool in chemistry has been underestimated or left aside.

Mechanochemistry is the chemistry that studies the chemical behavior of materials by mechanical effects without solvents or with negligible amounts of solvent in comparison with solution-based methods. By this way, being the reactives in high concentration and in close contact with each other, the mechanochemical activation by grinding or milling is very advantageous. The mechanochemical activator effect can be achieved by hand using hand mortar and pestle, by a mechanochemical reactor, by a mill reactor such as ball, vibratory, planetary and extrusion.

In the present invention, the choice of the compounds is such that, in addition of the interaction by mechanochemistry, a chemical reaction is promoted, since both reactants—a macrocycle (e.g. phthalocyanines, porphyrins) and a metal alkoxide—are prone to form complexes and are under free-solvent conditions. In addition, only two components are present in the mill for the mechanochemical synthesis and, optionally, further additives can be used depending on the further use.

The minimal use of solvent transforms mechanochemistry into a green alternative which is sustainable, environment-friendly and cost-effective.

In the present invention, the metal complex formation is promoted by both a mechanochemical reaction route and a metal alkoxide complexation route of two reactants: a macrocyclic/isoprenoid/linear tetrapyrrole compound and a metal alkoxide. In contrast to prior applications of mechanochemistry, the present invention is effective in the sense that it delivers products with high homogeneity and stability and smaller amounts of additives or hazardous by-products.

Mechanochemical mill or sonication is used as a tool for destroying the thick-walled cells of the microalgae such as Chlorella after harvesting by centrifugation. The processed algae are then dried (e.g. spray-dried). In order to inactivate some enzymes to avoid production of hazards for the health, the algae can be heated briefly to 100-130° C. Harvesting spirulina, for instance does not need maceration in order to be commercialized but washed to remove adsorbed salts. These mechanochemical steps are only the mechanical destruction of the cells in the biomass.

In Blue Biotechnology, in chapter 2,¹⁸ contains a detailed explanation of the careful culture maintenance in order to ensure the compliance and confidence of the consumer. Light, pH and temperature need to be carefully controlled to avoid contamination with other algae or bacteria (e.g. ciliates). The use of natural resources as a food or superfood for humans is still in its infancy due to the reluctance of the population to eat blue or green products considered always as toxic. Moreover, the use as cosmetics is hardly as a blue pigment.

Given the fact that most of the biomass from Chlorella and Spirulina are produced for human food and a small amount for cosmetics and the success of the food marketing is uncertain, since there are still some negative considerations regarding their toxicity and the use of some additives as stabilization ingredients in their formulation. It is an urgent necessity to look for other biomolecules or bio products from natural sources in order to extend the field of applications such as in painting, bioplastics manufacture.

Companies such as Cyanotech Corporation and Earthrise in the United States and GNT in the Netherlands—with partners in China or India, since most of the Arthrospira production occurs there—sold Spirulina as nutraceuticals or “superfood”. The company Earthrise and its partner DIC produce LINABLUE*¹⁹ which is a Spirulina extract prepared by the filtered aqueous extraction of the dried biomass of Arthrospira platensis. This extract must be free of impurities and negative to microcystin toxin according to FDA § 73.530 in order to be used as coloring confections, frozen products, ice creams and ready-to-eat cereals (excluding extruded cereals). Thus, this blue spirulina extract is a natural food colorant-phycocyanin and some additives such as trehalose and sodium citrate- and a functional food ingredient that is used in cold or frozen products that do not undergo heat processing, since it is not stable at high temperatures such as those used in extruding equipment. Earthrise¹⁹ as well as DIC²⁰ claim that “unlike artificial colorants, there is no blue tongue as LINABLUE® has no dyeing effect.” Given the fact that the upper surface of the tongue (dorsum) is a stratified squamous keratinized epithelium, it is understood that the LINABLUE® natural colorant does not dye keratinous material or other substrates since no dyeing effect is achieved.

Exberry claims to have the largest spirulina processing line in the world in Mierlo, the Netherlands. By contrast to the Linablue series, Exberry²¹ blues are trehalose-free but contain other carbohydrates or polysaccharides as food additives such as maltodextrin or sugar.

The use of one or other type of additives for a food colorant will attract more or less consumers in the food market but does not expand the market to other fields.

My invention is a strong opportunity to expand the market to other fields, for these industries such as dyes/pigments with dyeing effect with covering the whole spectrum for different kinds of substrates, processing and manufacturing of nature-based articles or plastics, among others.

The process of producing metal complexes of macrocycles and/or isoprenoids and/or linear tetrapyrroles of the present invention by the mechanochemical reaction (grinding or milling) of natural food colorant comprising macrocycles (e.g. chlorophyll) and terpenoids (β-carotenes) and linear tetrapyrroles (e.g. phycocyanin) such as Spirulina together with a metal alkoxide such as titanium alkoxide produces a titanium Spirulina intermediate or finished complex which can be used as a dye itself (with dyeing effect) of diverse substrates such a keratinous material and cellulose material. Furthermore, the color of the material produced are tuned by the deliberately selection of appropriate additives such as monomers or polymers. Similar results are obtained when other algae are used such as Chlorella.

Both companies producing the largest amount of Blue Spirulina claim that multicolors (purple and green) are possible since Linablue® can be combined with red, yellow and other natural colorants. With the primary colors, a physical change/another color that can be separated into their starting parts can be created.

The present invention provides another way to use the biomass or the biomolecules extracted from natural resources as a stable dye and pigments that replace existing dyes from non-removable sources different from the blue or green starting biomass. The generated colorants are produced by the solvent-free mechanochemical reaction of at least one macrocycle and at least one isoprenoid and at least one linear tetrapyrrole with a metal alkoxide. It is understood that there is no physical change of the components but a mechanochemical change/chemical reaction. The formation of colors is by the mechanochemical reaction of the reactants by grinding or milling and not the physical mixture of pigments or dyes such as those mixtures used by the painters. But these painters can use the intermediate and/or the finished product of the present invention to perform their creations in diverse substrates with or without additives.

The colorants with dyeing effect of the present invention are ready to be used as additives for other reaction such as those of polymerization.

The Undesired Discoloration

Macrocycles and/or isoprenoids and linear tetrapyrroles are often used as antioxidants or radical scavengers to protect products from discoloration. However, they suffer from color instability and are also prone to discoloration.

Chlorophylls as well as β-carotene are not exempt of these drawbacks. Hence, the limited possibilities of their industrial applications (e.g. as natural dyes). Discoloration can be caused by several factors such as exposure to daylight, indoor illumination, heat and air pollution.

The macrocycle/isoprenoid/linear tetrapyrrole metal complexes of the present invention are homogenous and stable in the presence of other additives, to storage and to light among others.

Macrocycles and/or Isoprenoids and/or Linear Tetrapyrroles as Colorants or Functional Dyes

Many macrocyclic compounds such as porphyrins have a wide gamma of colors, such as phthalocyanines and porphyrins. Copper phthalocyanines are after azo pigments, the most relevant for the pigment industry.

According to K. Hunger and M. U. Schmidt,¹⁰ only the copper, cobalt and nickel complexes of phthalocyanines have found industrial applications as pigments and/or dyes. However, the compound is employed almost exclusively in the form of copper phthalocyanine or their halogenated derivatives. Copper phthalocyanines suffer from instability in dispersion, since they tend to flocculate in binders and paints. This drawback is particularly apparent in combination with titanium dioxide in nonpolar media, as explained by P. Erik and H. Hengelsberg (2003) in chapter F.²² Moreover, it seems that sulfonation, halosulfonation and sulfonamide formation increase the stability, but it is not an economic and ecofriendly approach.

Although there are some methods to improve the stability to flocculation by using additives, the fastness properties of the existing phthalocyanines are only achieved through halogenation. Once again, the problem is not entirely resolved, since the halogenated metal complexes are even more expensive and not ecofriendly at all.

Given the fact that titanium oxide is the most common white inorganic synthetic pigment used commercially and in the paint industry and phthalocyanines are the second in commercial importance after the azo compound, they eventually will meet in the same paint formulation.

In the present invention, a stable colored metal complex of macrocycles (e.g. phthalocyanines and metal phthalocyanines) and metal alkoxide (e.g. titanium alkoxide) is produced by mechanochemistry (grinding and milling) under solvent-free conditions. Thus, the advantages of titanium-containing compounds are in the final product.

Macrocylces and/or terpenoids and/or linear tetrapyrroles are rich in healthy properties but also susceptible to oxidation, degradation or discoloration. Recently A. L. Focsan et al.²³ (2019) reviewed various approaches to overcome the drawbacks that limit the extended use of terpenoids such as carotenoids. The same drawbacks are also common in other natural products such as chlorohylls, or Spirulina. The encapsulation in supramolecular structures minimizes these disadvantages. Some techniques like encapsulation into nanoparticles, inclusion into cyclodextrins and into arabinogalactan are well known to enhance bioavailability in other compounds and can be also applied to carotenoids. Encapsulation in emulsions, liposomes and polymeric nanoparticles are also strategies to improve their application. Similar strategies have been considered with regard to other macrocycles and/or isoprenoids and/or linear tetrapyrrole.

Nevertheless, the complexation of macrocycles and/or isoprenois and/or linear tetrapyrroles with metal alkoxides by solvent-free mechanochemistry has not been used as an alternative to enhance the properties of macrocycles and isoprenoids/terpenois and linear tetrapyrroles until now.

In the present invention, a stable colored metal complex is produced without using solvents (or with a minimal amount of solvent as processing additive) by the reaction of macrocycles and/or isoprenoids and/or linear tetrapyrroles with metal alkoxides by mechanochemistry (grinding or milling). The colored metal complex is stable under different conditions such as daylight, storage and different formulations.

In the present invention, a macrocycle and/or an isoprenoid and/or a linear tetrapyrrole from different natural and industrial sources is used to react with metal alkoxides by mechanochemistry to produce homogeneous and stable colored metal complexes.

Metal Complexes of Macrocycles and/o Isoprenoids and/or Linear Tetrapyrroles as UV Sun Protection Filter or as UV-Absorber or as Material Protector for Further Processing, Such as Extractions or as Stabilization Mechanism.

UV filters are compounds that absorb or reflect the UV rays from the sun or artificial light. They prevent the premature damage and aging of the skin and hair.

Several synthetic filters used in sunscreens generate allergic reactions, and often synthetic UV filters are prohibited or their maximum concentration allowed is adjusted. The consumers' apprehension of using synthetic UV filters and the urge to find natural products have redirected the attention to the use of natural UV filter or absorbers. Macrocycles such as phthalocyanines and porphyrins and calixarenes have the potential to be used as UV filters.

In one embodiment of the present invention, the metal complexes of macrocycles and/or the metal complexes of isoprenoids and the metal complexes of linear tetrapyrroles obtained by mechanochemistry were used as sunscreen with concealer effect.

The micro algae, Arthrospira platensis (Spirulina or Arthrospira) and Chlorella vulgaris (Chlorella), contain high amounts of carotenoids, chlorophylls and phycocyanin (as well as high amounts of protein and aminoacids content). They contain many other beneficial nutrients such as β-glucans, vitamin B and minerals. They are cultured for commercial use in lakes or close/open ponds. They are sold as functional foods and they are generally regarded as safe (GRAS) by the European Food Safety Authority (EFSA).

Spirulina and chlorella are sources of macrocycles and isoprenoids and linear tetrapyrroles from nature. Therefore they are prone to capture or accumulate metals by the macrocyclic effect, at least. The common application of these kinds of microalgae are as human or animal nutrition, extraction of lectins, phycobiliproteins and bioactive peptides.

The use of alga or microalgae such as Spirulina and Chlorella as blue or green or blue-green pigments directly from the biomass without further extraction of their components is known to have several drawbacks that limit their use as dye/pigment. They are very heat- and light-sensitive. When they are used in processed food which is heated during the production, decoloration or bleaching of the pigment occur.

Thus, the use of algae/microalgae based pigments such as Spirulina or Chlorella is hardly as a nutrition or food colorant.

Although several way has been used to overcome this problem such as using antioxidants, e.g. polyphenols or carbohydrate/polysaccharide compounds, e.g. sorbitol or trehalose, the problem is still to be overcomed. Hence, the companies producing spirulina extracts highly recommend the use of this colorant as a food colorant for ice creams among others.

WO2015/090697A1²⁴ from BASF described the process of stabilization of phycocyanin blue pigments with polyphenols by dissolving between 0.1 to 0.5 g the polyphenol in an aqueous solution of phycocyanobilin or phycocyanin (0.5 g/L of solution). A stable complex was obtained up to 5 accelerated storage days. After 14 accelerated storage days, the solution comprising the complex of polyphenol and phycocyanin had lost 18% de blue coloration. WO2015/090697A1 is clearly a complexation of a polyphenol and phycocyanobilin/phycocyanin in aqueous solution. In addition, no metal alkoxide was used in the complexation, and there was no hint or suggestion of the use of a mechanochemical tool (grinding or milling) for the formation of the complexation. It can be concluded that this previous process from BASF was not a panacea.

Until now, the commercialization as pigment apart from coloring sugar-rich food (e.g. blue or green ice cream or syrups) is hampered, since it seems that these problems persist.

The present invention is a solvent-free reaction by mechanochemistry (grinding or milling of a macrocycle and/or an isoprenoid and/or a linear tetrapyrrole, such as Spirulina with a metal alkoxide. The present invention features e.g. the following
Solvent-free of with minimal amount of solvents that promote reactions e.g. mechanochemical reactions, never described before.
Mechanochemical tool, e.g. grinding, milling or extrusion, which promotes the formation of radicals prone to react.
Macrocyclic effect or template effect or whatever other effect that promotes the reactions in a confined environment.
Reactants such as macrocycles and/or isoprenoids and linear tetrapyrroles that are prone to react by a given effect with a metal alkoxide.
A metal alkoxide which is a versatile reagent with diverse physical properties that are conferred by the metal or by the chain or the substituents.

All of these features make the process for the production of the new compounds or complexes or supramolecular compounds or supramolecular dyes of the present invention a unique process.

The present invention improves the stability and the vulnerability to the environmental conditions of these microalgae pigments by the process of solvent-free mechanochemistry (grinding or milling) together with a material containing macrocycles and/or isoprenoids and/or linear tetrapyrroles such as Spirulina and Chlorella with a metal alkoxide. Thus far, there are no reports of using the mechanochemical process for the production of another colored material from alga/microalgae pigments e.g. in powder form, with enhanced properties such as stability in aqueous media, and at storage conditions in different physical forms.

In the present invention macrocycles and/or isoprenoids and/or linear tetrapyrroles from natural origin such as Spirulina powder are ground or milled together with a metal alkoxide without the use of organic or aqueous solvents or with a minimal amount of solvents for the production of another colored material which can be used as received as colorant or as intermediate for other physicochemical processes for the production of other high-value biomolecules.

Patent FR2929957A1²⁵ describes a process for the cultivation of cyanobacterium in an optimal medium to have abundant biomass, subjecting the biomass to stress with a salt and oxygen scavenger (NaCl and sodium sulfite solutions, i.e. induction medium) for 20 min to 1 hour, internalizing the divalent metal in the cyanobacteria medium at pH between 7 and 9.5, and incubating up to 2 hours at 20 to 30° C. after filtration and washing, extraction by conventional methods, grinding and precipitation with ammonium sulfate, followed by dialysis o ultrafiltration for removing the salt and unbonded metal and drying by atomization or lyophilization, a cyanobacteria heavily loaded with metal was produced. This same process was also performed for the phycocyanin. The carbonate and phosphate from the induction media have to be carefully removed in order to favor the introduction of the metal inside the cell.

Clearly, patent FR2929957A1 disclosed the procedure for internalizing metals into the cyanobacteria by inducing stress via salts and bleaching agents. After careful control of the pH by adding NaOH and the temperature of incubation, the final metal containing cyanobacteria was obtained after several hours of processing. Subsequently, several work-up processes were performed in order to get rid of the salts used.

In one embodiment of the present invention, a powder from natural origen comprising macrocycles and/or isoprenoids and/or linear tetrapyrroles ready to be used as superfood/food colorant, e.g. Spirulina biomass or blue spirulina extract/phycocyanine extract is ground together with a metal alkoxide to produce a metal Spirulina complex with another green or blue hue with enhanced properties. No control of pH was need since no water is added, not salts are added since metal alkoxides are not salts and, therefore, no purification/work-up steps are necessary to remove salts, among other advantages.

Keratinous Dyeing

One of the uses of the novel complexes formed in the present invention is as a hair dye either as a powder or as a formulation containing the same. The method of applying hair dye is also disclosed. The metal-complex dye produced in the present invention and the uses as a hair dye and the hair dyeing processes here presented are unique, and there are no records or suggestions in the prior art of the hair dye or dyeing techniques. The color itself is produced by the combination of two or three reactants or substances that confer to this dye the quality needed to be a good dye. The state of prior art from the hair dyeing literature or patents related to the present invention only concern the use of these four individual reactants, i.e. a macrocycle, an isoprenoid, a linear tetrapyrrole and a metal alkoxide, apart from each other in different hair dyeing systems.

Before Sir Perkin's discovery of the first synthetic dye in 1856, all dyes or pigments used were natural with or without mordants. After Sir Perkin's discovery, natural dyes were relegated due to their poor fastness to washing and to light. The hair dyeing and hair coloring industry is constantly growing, as is the awareness of the consumer and the professional personnel to use less risky and ecofriendly formulations and methods of application.²⁶

Briefly, there are three basic categories of hair colorant: temporary, semipermanent and permanent, depending on the durability of the color in the hair after washing. If the classification is done depending on the mechanism involved in the production of the color, there are oxidative and non-oxidative hair dyeing methods. Temporal and semi-permanent hair dyeing are non-oxidative and permanent hair dyeing is oxidative.

A conventional permanent or oxidative hair dye system comprises three types of reactives: primary intermediates, color couplers and oxidizers. The primary intermediates are capable of undergoing oxidation and subsequent development of color such as o- and p-phenylenediamine, o- or p-aminophenol or p-toloenediamine. The couplers such as m-diamines, m-aminophenol and phenols, do not produce any color when oxidized alone, but form dyes when oxidized in presence of primary intermediates. Thus, the couplers produce hair nuances to imitate a naturally looking color when they react with the initial oxidation product to give highly colored indo-dyes.²⁷ Thus, they react with the base intermediates to produce a dye. Oxidizers are usually hydrogen peroxide.

The most permanent (oxidative) hair colorants use aromatic amines such as p-phenylenediamine (PPD) in order to achieve long-lasting hair color, and it is supposed to be the only way to successfully color grey hair. It is a suspected carcinogen, alone or in combination with other components in the formulation, such as hydrogen peroxide, which produces other intermediates known to be mutagenic or carcinogenic.

The couplers, such as polyhydric phenols, e.g. resorcinol and pyrogallol, react with the intermediate products of the reaction with p-phenylenediamine to produce light brown colors.

WO2006/106366A1²⁸ discloses an oxidative hair dye comprising a hair dye, an organometallic and an oxidizing agent. The hair dye is a mixture of at least one developer and at least one coupler, such as those used in the conventional oxidative hair dye. The organometallic is a organotitanate such as tetraalkyltitanate (e.g. titanium alkoxide) and titanate chelates such as those commercially available and produced by Du Pont (preparation of many titanium chelates are reviewed in²⁹: “Titanium compounds, Organic”, Kirk-Ohmer Encyclopedia of chemical technology vol. 25).

Thus, developers and couplers are intentionally used to produce diverse colors in the final hair dye. Even the most preferred organometallic compound was aqueous diisopropyl di-triethanolamino titanate (Tyzor TE). Thus, a chelate that can release the triethanolamine upon hydrolysis and may lead to increase the pH as in the case of using ammonia. The abovementioned patent 66A1 describes that the organotitanate intensifies the color of the initial permanent hair dye system (having developer and coupler). Thus, the color is not produced by the organometallic compound as mentioned in the discussion of table 1 of 66A1. The authors affirm that the organotitanate used without the presence of additional oxidative hair dye does not act as a dye, i.e. imparting color to the hair.

In the present invention, the color is produced without the use of the conventional dye precursors (primary intermediates or couplers). It results from the reaction of a macrocycle and a metal alkoxide or the reaction of an isoprenoid and a metal alkoxide. Hence, amino-containing compounds and conventional oxidizer are non-essential for producing color.

In one system of the present invention, phthalocyanine—a macrocycle—and/or—carotene—an isoprenoid—are used for producing color, by reaction with the metal alkoxide either in the hair or applied to the hair, either after mixing the reactants or after the final powdered complex is obtained with a suitable functionalization of the surface, if desired. Only macrocycles and/or isoprenoids and/or linear tetrapyrroles are used instead of oxidation dyes (having primary intermediates and couplers).

It would be less risky for the consumer and the professional to deal with a hair dye product and process that does not contain aromatic amine or alkanolamine or any amino-containing compound to produce the color rather than those commonly used in oxidative hair dyeing. Even healthier would be the use of a hair dye system without oxidizer that forms hazardous and mutagenic intermediates such as those produced in conventional hair dye constellations.

In view of the high toxicity of the conventional oxidative hair dyes, it would be better to return to ancient practices where direct dyes or natural dyes were used for coloring keratinous materials such as henna (lawsone), walnut tree or shells (juglone), chamomile (apigenin), logwood (hematin). Carotenoids are less used as natural hair dye due to fugitive properties: it fades under air; it is unstable at different pH and temperature conditions, as explained above.

Natural dyes are also employed as combinations of several natural dyes and mordants such as salts of aluminium, chromium, iron and tannins. Metal salts are used for fixation of the dyestuff and increasing the fastness properties. In particular iron mordants lead to color differences with darker shades. However, many of these salts precipitate in the hair dyeing formulations or are water-soluble and drip out the hair when weathering or sporting.

Conventional hair dyes, such as those using henna, are mixed with metal salts as mordants and have several drawbacks, such as loss of color due to water or shampooing. Henna is only adhered to the hair and not absorbed. Even peroxides and arylamines are added to the henna formulations to improve the dyeing behavior. A major challenge of natural dyeing is that only very few sources for red and blue are available.

Commonly, carotenoids are used in combination with other natural dyes to produce several colors such as Carthamus tinctorius (safflower) that gives a scarlet, with indigo to produce olive green. These are beautiful as textile dyes, but they do not withstand washing even after using salts as mordant. For hair dyeing, the process is even less suited due to the necessary mild and healthy conditions that the human keratinous dye requires.

The extent of the applicability of natural dyes in the dyeing industry is limited predominately due to the small range of colors and laborious and time-consuming methods of application.

The use of metal complexes as dyes owes its origin to the mordant dyeing. In regard to metal-complex dyes Ullmann's Encyclopedia of Industrial Chemistry describes extensively the use of metal chelation of azo/azomethine dyes. The anionic 1-2 chromium complex with salicylic acid, sodium diaquabi[salicylato(2-)O1,O2] chromate(1-), is generally used as a precomplex to synthesize 1-2 chromium complexes of azo dyes in aqueous solution at high pH for introducing hydrophilic groups in the complex with outstanding wet- and light fastness. This is the so-called chromium(II) salicylic acid method of chromatation. The complex formation on the salicylic acid group has little influence on shade and lightfastness contrary to the azo compounds.

As a result, many formulations of natural hair dyes are combined with oxidative hair dyes to achieve higher color intensity, stability and durability. Nearly all chromophore systems commonly used in synthetic dye chemistry (nitro, azo, anthraquinone, triphenylmethane, azomethine) are used. Thus, toxic colorants are generally masked with the name “natural,” while containing all reactants commonly found in conventional oxidative hair dye.

Thus, colorants with enhanced properties that combine the benefits of a natural colorant with green chemistry synthesis and with effective and healthier properties are needed not only in the dyeing process of different substrates but also in the industry. Moreover, the addition of a metal alkoxide never used for dyeing with the formation of a color that is difficult to obtain in nature is disclosed in the present invention.

In the present invention, macrocycles and/or isoprenoids and/or linear tetrapyrroles (e.g. natural dyes such as chlorophylls, β-carotene and bilirubin) are used for the formation of color with or without other macrocycles or other natural colorants. The reaction is produced either into or onto the hair by reacting with a metal alkoxide. This hair dyeing system is novel and, when applied to the hair by several methods, imparts a semi-permanent or temporary color.

Surface Modification of Macrocycles and/or Isoprenoids and/or Linear Tetrapyrroles Involving the Use of Metal Alkoxides

Ultrahydrophobic or superhydrophobic surfaces are water-repellent similar to those of lotus or water lily leaves, which are extreme difficult to wet. Artificial superhydrophobic materials or surfaces are a bio-inspiration. There are several surface modification methods for changing the wettability of a surface such as chemical treatment, vapor deposition or etching, plasma treatment, laser treatment, electrospinning. They have the limitation that the film, generally a polymer, is easily cracked. In addition, contamination, e.g. powder deposited in the liquid drop, reduces the hydrophobicity of the surface.

It is another object of the present invention to provide a surface modification method by using grinding or milling of a macrocycle and/or isoprenoid and/or linear tetrapyrrole together with a metal alkoxide in a suitable surface such as porcelain, glass or wood.

The hydrophobic surface of the present invention can also be used to generate big compartments of water surrounded by the amphiphilic metal complex produced in the present invention.

Catalyst for Reactions Such as Polymerization

The present invention comprising the synthesis of metal complexes to be used as a catalyst for several reactions or polymerization is characterized by the one-step production of an ecofriendly catalyst for reactions of polymerization. Transition metal macrocycles are already used to substitute the toxic mercury catalyst for the polyurethane formation such as titanium, copper and niobium.

In the present invention, a macrocycle and/or an isoprenoid and/or a linear tetrapyrrole (e.g. chlorophylls and carotenoids) which are of low or no toxicity and are used to start the direct complexation with a metal alkoxide by mechanochemistry for further uses as catalysts.

Artificial Phothosynthesis

As previously mentioned, nature uses metal macrocyclic ligands in biological systems such as photosynthesis. Such systems include chlorophyll, haemoglobin, and vitamin B₁₂. In these entities, the metal ion is held firmly in the macrocyclic cavity and, by this way, the biological role of these entities is not diminished by other reactions (e.g. competing demetallation processes).

In the present invention, metal-containing macrocycles and/or isoprenoids and/or linear tetrapyrroles are subjected to the mechanochemical reaction (grinding or milling) with metal alkoxides to produce stable metal or bimetallic complexes of macrocycles and/or isoprenoids and/or linear tetrapyrroles with enhanced properties. Thus, the macrocyclic effect of the metal macrocycle and/or metal isoprenoid or metal linear tetrapyrroles are used to generate other molecular assemblies.

These novel final assemblies of the present invention are a tool to produce new complexes/dyes/pigments that might be used for the artificial photosynthesis since they resemble the natural process of photosynthesis in which several dyes are present and a broad gamma of colors can be produced including near-black color or black colors.

Method of Producing Metal Nanocomposites

There is a great verity of physical, chemical and physicochemical methods to incorporate metals into other materials. Pomogailo A. D and Kestelman V. N³⁰ (2005) reviewed the techniques to produce macrocomplexes, or metallopolymers or nanocomposites or metallopolymer nanocomposites.

None of these physical, chemical or physicochemical methods use the method of producing the metal containing composites of the present invention by solvent-free mechanochemical synthesis of metal complexes using metal alkoxide as the metal precursor.

### DETAILED DESCRIPTION OF THE INVENTION

This invention covers a process for the synthesis of metal complexes of macrocycles from at least one macrocycle and at least one metal alkoxide by mechanochemistry (grinding or milling) under solvent-free conditions. Similarly, this invention covers a process for the synthesis of metal isoprenoid complexes from at least one isoprenoid and at least one metal alkoxide by solventless mechanochemistry. In the same way, this invention deals with the mechanochemical synthesis of metal complexes of linear tetrapyrroles by reacting at least one linear tetrapyrrole with at least one metal alkoxide by grinding or milling. This invention covers a solvent-free mechanochemical synthesis of a stable metal complex by reacting at least one macrocycle and/or at least one isoprenoid and/or at least one bilane with a least one metal alkoxide. If a metal macrocycle (e.g. chlorophyll) or a metal isoprenoid and/or metal linear tetrapyrrole is used instead of the metal-free reactant to react by grinding or by milling with the metal alkoxides, other kinds of assemblies are generated that resemble natural processes such as photosynthesis.

In particular, the present invention deals with the mechanochemical synthesis of metal complexes by using e.g. porphyrin and/or phthalocyanine/copper phthalocyanine and/or chlorophyll (magnesium or copper and/or calixarene and/or squalene and/or Spirulina and/or and/or β-carotene and/or squalene and at least one metal alkoxide as starting materials.

Macrocycles

The macrocycle ligand has the ability to coordinate with most elements which is a feature that distinguishes it from other organic ligands. Dodziuk H.³¹ (2002) described most interesting macrocyclic ligands. Some of the macrocycles fall clearly into the domain of host-guest chemistry (e.g. cyclodextrines). Others macrocycles such as fullerenes can not only host ions or molecules inside the cage but also play the guest roll of being buried inside a cage, capsule or host.

All macrocycles and their derivatives are interesting ligands because they are good hosts not only for metals but also for neutral molecules. The feature of macrocycle chemistry is the tendency to form tri or tetramer complexes with a metal for most transition and main group elements.

The at least one macrocycle compound, either synthetic or the natural origen, containing oxygen, nitrogen, sulfur or phosphorus donors/elements as replacement of other skeletal atoms (e.g. nitrogen or carbon en porphyrins) and other modifications, substituted or unsubstituted, with planar or non-planar structures, with or without containing a complexed metal ion is selected from:
polyaza macrocycles (simple polyaza macrocycles, cyclidenes, sepulchrates, bis-macrocylces, expanded porphyrins), e.g. cyclam or polyaza criptate;
Simple and multi-ring aromatic compounds, e.g. anullenes, pyrene, coronene, ovalene, perylene, phenanthrene, kekulene, hexahelicene, graphite, graphene or fullerene;
Tetrapyrroles and their relatives (β-substituted porphyrins, meso-substituted porphyrins, metal porphyrins, ring-expanded porphyrins, ring-contracted porphyrins, reduced porphyrins) e.g. porphyrins, meso-tetraphenylporphine; chlorophylls (a, b, c and d), chlorophyllin, bacteriochlorophylls; chlorins; bacteriochlorin; carotenoporphyrins; corrin; corrole; sapphyrin; heme; hemochrome; hemin or hematin;
Fused macrocyclic tetrapyrrole systems, e.g. phthalocyanines or metal phthalocyanines, e.g. copper phthalocyanines, titanyl phthalocyanine, tetrabenzoporphyrin, mono, bis, tris and polyphthalocyanines; polythia, polyphospha or polyarsa macrocycles;
Mixed donor macrocycles, e.g. cryptands, compartmental ligands, catenanes or rotaxanes;
Polyoxa macrocycles or crown ethers, e.g. polyether macrocycles, lariat ethers, spherands or hemispherands;
Calixarenes; pillarenes; resorcinarenes; cavitands; carcerands;
Terpenoid macrocycles, e.g. taxol, rapamycin, ascomycin or tacrolimus;
Alkaloid macrocycles, e.g. Trabectedin;
Macrolactones; macrolides; cardenolides; bufadienolide, e.g. erythromycin;
Peptide or protein-based macrocycles (globin), e.g. hemoglobin, myoglobin, picket-fence porphyrin complex, or hemeprotein;
Fullerene macrocycles and their related materials, e.g. endohedral or exohedral fullerenes, graphenes, graphite, or carbon nanotubes;
Organic zeolites;
Dendrimers;
Polyketide macrocycles or macrolides;
Perylene-based macrocycles;
Cyclophanes, e.g. paracyclophanes;
Cyclotetraicosaphenylene;
Cyclodextrins;
Cucurbiturils;
Vitamins and derivatives, e.g. vitamin B₁₂ (or cobolamin);
Macrocyclic bile acid, e.g. cholic acid, chenodeoxycholic acid, deoxycholic acid and lithocholic acid;
Other naturally occurring macrocycles displaying a variety of biological activities (immunosuppressant, antibiotic, anticancer, antifugal, ACE inhibitor), e.g. FK-506, tetracycline, aminoglycoside streptomycin, paromomycin, vancomycin, epothilone B, geldanamycin gentamicin, K-13, amphothericin B, amoxicillin or clarithromycin;
Formulations or compounds containing macrocycles including nanoparticles, liposomal encapsulation, phospholipid complexes, emulsions, capsules, tablets and powders, either used alone or in combination with other compounds, such as medicines, antibiotics, polyphenols, alkaloids (piperine), carbohydrates (monosaccharides, oligosaccharides and polysaccharides), aminoacids, peptides, proteins; herbal preparations, such as extracts or tinctures containing said macrocycles;
Isoprenoid/terpenoid-modified macrocycles, e.g. cytoporphyrin;
Mixtures or modifications thereof.

F. Davis and S. Higson³² presented a detailed list of macrocycles, their construction and their chemistry, that may be used as a source of macrocycles for the metal complexation by mechanochemical grinding of the present invention.

W. Herbst and K. Hunger¹⁰ reviewed the syntheses of some macrocycles (e.g. phthalocyanines and metal phthalocyanines).

Macrocycles may also be obtained by in situ synthesis prior or during the complexation of the present invention.

Isoprenoids

The at least one isoprenoid/terpenoid, either synthetic or of the natural origin, containing oxygen, nitrogen, sulfur, fluorine or phosphorus donors/elements as replacement of other skeletal atoms (e.g. fluorine in retinoids) and other modifications, substituted or unsubstituted, with or without containing a complexed metal ion is selected from:
Carotenoids either carotenes or xanthophylls (hydrocarbons, alcohols, glycosides, ethers, epoxides, aldehydes, acid and acid esters, ketones, esters of alcohols, apo-carotenoids, nor- and seco carotenoids, retro-carotenoids and retro-apo-carotenoids), e.g. acyclic carotenes, lycopene, carotenes (α, ψ, β, ε, γ, κ, ϕ, χ), capsanthin, lutein, criptoxanthin, zeaxanthin, neoxanthin, violaxanthin, flavoxanthin, astaxanthin, bixin, crocetin, crocin, fucoxanthin or iridoids;
Terpenoids (hemiterpenoids, monoterpenoids, sequiterpenoids, diterpenoids, sesterterpenoids, triterpenoids, tetraterpenoids, polyterpenoids), e.g. isoprene; prenols; dolichol; polyprenols; steroids; sterols/phytosterols, e.g stigmaterol; carotenoids; ginkgolide; bilobalide; citral; menthol; camphor; salvinorin A; cannabinoids; farnesol; carvone; eucalyptol or squalene;
Macrocycle-modified isoprenoids/terpenoids, e.g. carotenoporphyrins or carotenofullerenes;
Cyclic monoterpenoids (iridoids), e.g. genipin, geniposide, aucubin or catalpol;
Retinoids (retinol, retinal, retinoic acid, retinyl esters, nor- and seco retinoids, retro-retinoids), e.g. vitamin A;
Vitamin and derivatives, e.g. vitamin K;
Tocopherols, e.g. α-tocopherols or vitamin E.
Peptide or protein-based isoprenoids/terpenoids, e.g. prenylated proteins;
Formulations or compounds containing isoprenoids/terpenoids including nanoparticles, liposomal encapsulation, phospholipid complexes, emulsions, capsules, tablets and powders, either used alone or in combination with other compounds, such as medicines, antibiotics, polyphenols, alkaloids (piperine), carbohydrates (monosaccharides, oligosaccharides and polysaccharides), aminoacids, peptides, proteins; herbal preparations, such as extracts or tinctures containing the isoprenoids/terpenoids, e.g. paprika oleoresin;
Mixtures or modifications thereof.

Linear Tetrapyrroles and their Relatives

The at least one linear tetrapyrrole, either synthetic or the natural origin containing oxygen, nitrogen, sulfur, fluorine or phosphorus elements as replacement of other skeletal atom (e.g. oxygen in phycoerythrobilin) and other modifications, substituted or unsubstituted, with or without containing a complexed metal ion is selected from:
linear tetrapyrroles (bilanes, bilins, bilenes, biladienes), e.g. biliverdins, mesobiliverdins, bilirubins, mesobilirubins, urobilins, stercobilins, urobilinogens, phycoerythrobilin, phycocyanobillin/phycobiliverdin, phycoviolobilin, secocorrin or phycourobilin;
Phycobilins, e.g. phycoerythrobilin, phycocyanobillin, phycoviolobilin or phycourobilin;
Protein-pigment complexes, e.g. phycocyanin, phycoerythrin phycoerithrocyanin, allophycocyanin or phytochromobilin;
Relatives to linear terapyrroles compounds, e.g. linear tripyrroles (e.g. tripyrrin or reduced tripyrrin); dipyrroles, (e.g. dipyrrin (formerly dipyrromethene), dipyrrinl-(10H)-one (formerly pyrromethenone) or dipyrromethane (formerly dipyrrylmethane).
Mixtures or modifications thereof

Macrocycles and/or Isoprenoids and/or Linear Tetrapyrroles

The at least one macrocycle and/or the at least one Isoprenoid and/or the at least one linear tetrapyrrole as extracts in any form, tinctures, essential oils or powders/biomass (from each part of the natural source or the whole source) either synthetic or of natural origen, either with natural or delivered induced modification, either natural or synthetic products after recombinant techniques or physicochemical techniques, is selected from:
Terpenoids, e.g. from turmeric, ginger or rubber tree;
Cannabinoids, e.g. myrcene, β-cariophyllene or limonene;
Iridoids (from Gentianaceae, Rubiaceae, Ericaceae, Valerianaceae) e.g. genipin;
Coenzymes, e.g. ubiquinone (Coenzyme Q₁₀);
Protein-based macrocycles/isoprenoids/linear tetrapyrroles, e.g. phycocyanin or hemoglobin;
plants of genera: Hevea, Landolphia, Taraxacum, Palaquium, Amaranthus, Zingiber, Vitis, Citrullus, Citrus, Coriandrum, Cotinus, Euphrasia, Lavandula, Verbenacea, Illicium Carum, Mentha, Calendula, Bursera, Artemisia, Vachellia, Cinnamomum, Eucalyptus, Glycyrrhiza/liquorice Syzygium, Betula, Backhousia, Leptospermum, Ocimum, Solanum, Helianthus, Cannabis, Lupinus, Brassica, Crataegus, Curcuma, Gardenia, Crocus Lawsonia, Indigofera, Genipa, Oenothera, Lespedeza, Passiflora, Hamamelis, Theobroma, Coffea, Chamaemelum, Quercus, Capsicum, Molva, Bixa, Tagetes, Cynara, Glycine, Asperula, Angelica, Hieracium, Ammi, Melilotus, Aesculus, Lithospermum, Solidago, Origanum, Camellia, Schisandra, Hibiscus, Rosa, Ribes, Acacia, Bactris, Rhus, Gingko, Juglans, Moringa, Lavandula or Persea;
Algae(brown algae (e.g. kelp), red algae (e.g. Gracilaria, Porphyra), green algae (e.g. Haematococcus pluvialis, B. braunii)), e.g. astaxanthin;
Microalgae (cyanobacteria and eukaryotic algae), e.g. Arthrospira/Spirulina (e.g. Arthrospira platensis, A. fusiformis, A. maxima), Chlorella (e.g. vulgaris, pyrenoidosa), Dunadiella salina, Aphanizomenon Flos-aquae), e.g. blue Spirulina extract, Spirulina biomass/powder, Chlorella biomass/powder, asthaxanthin powder, β-carotene or chlorophylls;
Fungi (e.g. Aspergillus, Trichoderma, Penicillium, Bipolaris), e.g. antibiotics, phytohormones, metacridamides, trichothecenes or macrocyclic polylactone;
Bacteria (from E. coli, Streptomyces Hygroscopicus), e.g. aromadendrene, geldanamycyn;
Yeast (genetically modified), e.g. farnesene;
Animals or humans (e.g sterols or steroid hormones, pheromones (e.g dendrolasin, iridomyrmicin), squalene, lanosterol, cholesterol), e.g, Euphausia pacifica from krill, Euphasia superba from krill, Pandalus borealis from shrimp;
Mixtures or modifications thereof.

The modified macrocycles and/or isoprenoids and/or linear tetrapyrroles can be selected from those which have been subjected to the complexation procedure of the present invention. In addition, the macrocycle compounds or isoprenoid/terpenoid compounds and/or the linear tetrapyrrole compounds of the present invention include those materials that are subjected, prior to the complexation of the present invention, to another kind of chemical, biochemical, enzymatic, genetic or physical procedure or complexation. As further presented in this invention, the encapsulation of macrocycles or carotenoids is a strategy to enhance their properties. Thus, macrocycles or isoprenoids or linear tetrapyrroles which are modified or protected are also used as a source of macrocycles or isoprenoids or linear tetrapyrrolesin the present invention.

The plant, alga or fungus extract is a derivated extract from the whole plant or part of the plants such as flowers, leaves, stems, fruits, bark, roots, seeds, and resin obtained by any kind of extract process.

Microalgae are an excellent natural source of of many carotenoid and chlorophyll pigments. Chlorella and Arthrospira [“Spirulina” ] are commercialized as healthy foods. Dunaliella Salina as source of β-carotene and Haematococcus pluvialis s source of astaxanthin. The thraustochytrid Aurantiochytrium as well as the green alga B. braunii are source of squalene as well. Squalene in cell is an intermediate in the biosynthesis of cholersterol and other steroids.

In particular, the term Spirulina is the biomass of cyanobacteria (blue-green algae) which was formerly classified as a genus Spirulina. The species are Arthrospira platensis, A. fusiformis and A. maxima.

In the present invention, the term macrocycle and isoprenoid or linear tetrapyrrole refers also to extracts of materials containing these compounds which have been subjected to the complexation procedure of the present invention. In addition, the macrocycle and/or isoprenoid/terpenoid compounds of the present invention include polyphenols that are subjected, prior to the complexation of the present invention, to another kind of chemical, biochemical, enzymatic, or physical procedure or complexation. As further presented in this invention, the encapsulation of macrocycle and/or isoprenoid and linear tetrapyrroles is a strategy to enhance their properties. Thus, macrocycle and isoprenoid and linear tetrapyrroles which are modified or protected are also used as a source of polyphenol in the present invention.

Macrocycles and isoprenoids and linear tetrapyrroles have several health benefits for humans and animals due to several properties, including antioxidant, anti-inflammatory, cardioprotective and neuroprotective functions. Besides, macrocycles and/or isoprenoids and/or linear tetrapyrroles are anti-bacterial, anti-viral and anti-fungal. These capabilities of these compounds are key to their use for treatment of several diseases, in food-processing and for anti-aging purposes in various cosmetic and pharmaceutical formulations. However, some macrocycles and/or isoprenoids and linear tetrapyrroles such as chlorophylls, carotenes, Spirulina or Chlorella are sensitive to several environmental factors such as light and heat and may degrade rapidly under water, air or storage conditions.

As previously mentioned, their use of alga/microalga-based pigments such as spirulina or chlorella as food or pigments is hampered by the instability of the color under environment, pH, heat.

The present invention provides a new synthetic approach to stabilizing macrocycles and/or isoprenoids and/or linear tetrapyrroles by complexation with metal alkoxides by mechanochemistry (grinding or milling) with an excellent stability e.g. to environmental and storage conditions. These novel metal complexes can be further encapsulated or processed, e.g. by miniemulsion polymerization for further applications.

Metal Alkoxides

According to IUPAC,¹ the term alcoholates is synonymous of alkoxides. Alcoholates should not be used for solvate derivates from an alcohol such as CaCl₂·nH₂O, for the ending—ate often occurs in names for anions.

Metal alkoxides³³ or metal alcoholates have the formula M(OR)ₓ, where M is the metal (or non-metal or other cationic species), R is an organic radical and x corresponds to the valency of the metal M. The metal alkoxide can be either in solid or liquid form. They are produced from almost any metal of the periodic table of the elements. Both metal and radical confer properties to the alkoxide. The metal provides the electronegativity, whereas the radical provides the acidity and the ramification. Thus, metal alkoxides are so versatile and diverse that they can be either water-soluble or water-sensitive. One of the advantages of alkoxides is that they only form their parental alcohol as a by-product. The health hazard of metal alkoxides depends on the metal they contain and the alcohol they produce after hydrolysis. M(OR)ₓ or [M(ORₓ)]ₙ is a metal alkoxide or heterometallic alkoxide such as mixed alkoxide, mixed halide-alkoxides or bimetallic alkoxide (double alkoxide), or polymeric metal alkoxide or oxo metal alkoxide, metal aryloxide or bi-, tri-, and tetrametallic alkoxides or adducts with neutral ligands where
(a) M is one or more elements from the elemental periodic table, preferably titanium, zirconium, hafnium, vanadium, aluminium, germanium, silicon, niobium, lithium, tantalum, zinc, magnesium, antimony, indium, gallium, copper, holmium, tin, lanthanum, erbium, barium, gadolinium, yttrium, tantalum, dysprosium, cobalt, tellurium, lead, bismuth, calcium, cerium, iron, strontium, molybdenum, tungsten, neodymium, nickel, samarium, europium, osmium, praseodymium, boron, sodium, potassium, thallium, scandium, chromium, manganese or mixtures thereof;
(b) R is an organic radical such as methoxide, ethoxide, propoxides (n- and iso-), butoxides (n-, iso-, sec-, and tert-), amyloxides (n-, sec-, tert-) and neopentyloxides, aryloxides;
(c) x corresponds to the valency of the metal M and
(d) n corresponds to the degree of molecular association.

The term heterometal alkoxide with bi-, tri-, and tetrametallic alkoxides (as explained in the corresponding chapter in Alkoxo and Ariloxo Derivatives of Metals by D. C. Bradley et al.), such as bimetallic alkoxides MₙM′ₘ(OR)ₚ is adopted here since their structure and physicochemical properties belong to the same type as those of homometallic ones. A metal alkoxide or a heterometal alkoxide is not a salt, since they do not proceed from the reaction of an acid and a base neutralizing each other.

Polymeric metal alkoxides [M(OR)ₓ]ₙ are the product of partial hydrolysis or thermolysis of M(OR)ₓ, where n corresponds to the degree of molecular association.

Adducts of metal alkoxides with ligands such as M(OR)ₓ·mL, where m is the composition of the solvates, can also be used.

Optionally, the metal alkoxide used in the present invention can be prepared in situ by reaction of alcohols with metals, with metal hydroxides, with metal halides, with metal amides or metal alkoxide of other alcohols or mixed halides alkoxides or by alcoholysis and transesterification reaction and other methods (alkoxides, metal in Kirk-Othmer Encyclopedia of Chemical Technology, vol. 2).

All macrocycles/isoprenoids/linear tetrapyrroles can be modified by undergoing different reactions leading to analogue compounds (e.g. endohedral fullerenes) that can be further used as modified macrocycles for the complexation of the present invention. Nevertheless, until now, the use of the solvent-free mechanochemical complexation (by grinding or milling) of macrocycles and/or isoprenoids and/or linear tetrapyrroles with metal alkoxides to produce metal complexes of macrocycles and/or isoprenoids has been unknown.

The metal macrocycle complex synthetized in the present invention is the product of the complexation between a macrocycle and a metal alkoxide promoted by mechanochemical synthesis to assure homogeneity of the final product.

The metal isoprenoid complex synthetized in the present invention is the product of the reaction of an isoprenoid with a metal alkoxide.

Macrocycle and/or isoprenoid and/or linear tetrapyrrole may be mixed before the reaction with the metal alkoxide. In this way, even more different complexes and colors/materials are obtained.

In the present invention, the mechanical grinding is preferably achieved by a simple hand mortar and pestle. Various mechanical deformation methods, well known in the prior art, can be used such as mechanical mortar and pestle, high speed milling, ball milling, attrition milling and planetary milling.

The process of the invention may comprise the following steps:
1. At least one metal alkoxide is added to the macrocycle and/or the isoprenoid/terpenoid and/or linear tetrapyrrole (or vice versa) in the desired stoichiometric molar ratio metal alkoxide to macrocycle and/or metal alkoxide to isoprenoid and/or metal alkoxide to linear tetrapyrrole, such as between 1/1000 to 1000/1 or vice versa. All reactants are homogeneously mixed/macerated before step 2. In some cases where the molecular weight is not exact known, e.g. in the case of Spirulina, weight ratio metal alkoxide to macrocycle and/or isoprenoid and/or linear tetrapyrrole between 1/1000 to 1000/1 may be used.
   (a) Optionally, depending of the further use of the metal macrocycle/isoprenoid/terpenoid/linear tetrapyrrole complex as a sunscreen, dye or colloid, an amount of additive between 0.01 wt % and 50 wt % of the total weight of both reactants is added. The additive compound is selected either as a single compound or a combination of two or more compounds the group of further macrocycles, further isoprenoids/terpenoids, further linear tetrapyrroles, further metal macrocycle complexes, further metal isoprenoid/terpenoids complexes, further metal linear tetrapyrroles complexes, further metal complexes, polyphenols/antioxidants, β-diketones, fullerenes and related materials (e.g. carbon nanotubes, graphene, graphite), monomers (e.g. maleic anhydride), synthetic polymers, natural or modified polymers (e.g. lignin), polysaccharides (e.g. cellulose, glucan, trehalose, carrageenans), DNA and RNA, solvents, fatty oils, phenolic acids, fatty alcohols, organic acids, vitamins, aminoacids, lipids, proteins, carboxylic acids, synthetic or natural colorants, wetting agents, swelling agents, penetrants, pH regulators, surfactants, perfumes, thickeners, milling adjuvants, salts, oxides or water.
   (b) Optionally, the additive is added to the reactants taking account of the compatibilities.
   (c) Optionally the additive is added simultaneously with the reactants.
2. The abovementioned mixture is ground by hand or mechanically (by hand or mechanical mortar and pestle or by ball milling), so that the mechanochemical reaction occurs between the two reactants. Several mechanochemistry methods can be used, such as hand and mechanical mortar and pestle, ball milling, attrition milling, planetary milling or extrusion. The mechanochemical reaction gives a colored, homogeneously and finely dispersed powder material. The color in the final powder depends on the metal used and the radical of the metal alkoxide as well as the macrocycle, the isoprenoid and linear tetrapyrrole ligand.
3. Optionally the mechanochemical reaction (grinding or milling) may be carefully tuned whereby different physical forms of the final material might be obtained. By controlling the additives added, the time and the speed of the grinding and milling, materials in diverse physical forms are obtained.
4. Optionally, the additive is added after the complex is formed.
5. The abovementioned mixture may be let to rest (such as for 1 hour).
6. The resulting material (e.g. finely dispersed and colored powder or homogeneous paste or colloid) is ready to be used in food, cosmetics, pharmaceuticals, paints, and other applications.
7. Optionally, the ground mixture is left as an intermediate state—before the finished metal complex form is obtained—for further reactions or processing.
8. The physical form of the colored material both the intermediate metal complex and the finished metal complex, such as powder, presscake, granule, chip or lake, liquid, liquid dispersion, colloids, paste, liquid crystals or flush color is tuned by the conditions of the mechanochemical reaction such as the type of the reactants, the proportion of the reactants, the temperature, the milling/grinding time, the speed, the ball/weight ratio, the milling media, the presence or the absence of additives. Preferably the metal complex of the present invention is in powder or colloidal form.

The macrocycle and the isoprenoid and the linear tetrapyrrole and the metal alkoxide act as reactive compounds prone to react by the macrocycle effect, the chelating effect, the host-guest effect, hydrophobic effect, confinement effect and any other plausible effect. The mechanochemical synthesis enhances and promotes the reaction of all reactive compounds and provides the desired colored product or product precursor for further uses such as sunscreens. All reactive compounds used for the process of the present invention can be either in solid or liquid forms under the conditions used for the mechanochemical synthesis. In addition, one or more or even all reactive compounds for the process of the present invention can be used in gaseous state under the conditions used in the mechanochemical synthesis. Preferably, at least one other compound is either solid or liquid under the conditions used in the mechanochemical process.

The mechanochemical route of synthesis is a direct synthesis that uses either manual or mechanical milling or grinding to initiate or facilitate the process in any physical state. Either solid-solid-solid synthesis or liquid-liquid-liquid synthesis or gas-gas-gas synthesis or combinations thereof without solvent or with minimal amounts of solvent is used to promote mechanochemical reactions. The most important advantage of mechanochemistry is the solvent-free synthesis or near solvent-free synthesis with only small amounts of solvents (i.e. less than 10 wt % based on the total of weight of the reaction mixture), the so-called liquid-assisted grinding. According to K. Tanaka (2009),⁴ the term solvent-free refers to the stoichiometric application of solid or liquid reagents with less than a 10% excess of a liquid or soluble reagent and/or less than 10% of a liquid or soluble catalyst. Minimal amount of solvent implies that no solvent is a priori and deliberately added to the reaction that could require solvent-consuming purification steps after the reaction.

Solvent-free organic synthesis is eco-friendly and obviates the necessity of further steps of solvent evaporation and recycling of the solvent. In addition, the amount of hazardous by-products that can interact with the solvent is decreased.

In contrast to mechanochemistry, the traditional production of metal macrocycle complexes by solution-based methods uses substantial amounts of solvents, generally organic such as toluene or benzene in order to promote the reaction, to remove the byproducts produced during the reaction and to recover the product of the reaction. Consequently, the solvent has to be removed by different methods such as distillation, distillation under reduced pressure or vacuum extraction. This process is tedious and involves careful handling of toxic compounds. In the present invention, since both the metal alkoxide and the macrocycles and carotenoids are sensitive to solvents, it is beneficial to let them react only by solventless mechanochemistry.

Mechanochemistry can promote reactions quickly and in large quantities. The effectiveness of the mechanochemical reactions depends on the chemical and the mechanical properties of the agents or reactants. Mechanochemical phenomena may lead to the activation of strong covalent chemical bonds by the presence of an external mechanical force. However, more labile non-covalent bonds may also be activated, e.g. supramolecular materials.

Although the theory of the mechanochemical reactions is still in its infancy and, so far, there is not a general theory for mechanochemical reactions, some possible phenomena may involve:

Formation of active surface radicals; modification of physicochemical properties; enhancement of reactivity due to stable changes in the structure; enhancement of effectivity in the solid phase than in the liquid phase.^{34,35}

The mechanochemical reactions of the process of the present invention can be made by various methods.

Prior to starting the mechanochemical process, a detailed control of the form of the reactive compound should be effected. If both reactive compounds are grove solids, they can be ground separately before the mechanochemical process begins. Optionally, if possible, a slight increase of the temperature for allowing producing a homogeneous mixture would be convenient. The starting materials are combined at slow speed and then ground together at higher speed. The grinding can be done under dry or wet conditions. Dry grinding is preferred. However, in case wet grinding is required, inert grinding aids that do not react with either the reactant compound or the final product are preferred.

The energy of the grinding process can be varied within wide ranges. Low energy ball-milling, attrition milling, vibratory milling and similar low energy grinding processes known in the art of grinding are preferred over high energy milling processes, since the use of a grinding medium at high energy can produce wear and, thereby, contaminate the reactant and the product obtained. However, when high energy milling is needed, high energy ball milling, high energy planetary milling and similar can be used. Grinding media that do not react with the reactant compound and product are preferred and can be agate or similar materials. To increase the reactivity of the reactant compounds or to induce melting of one or more reactants if need be, the milling process can be carried out at high temperatures or the reactive compounds can be pre-heated prior of the start of the milling process.

Additives such as cutting, milling and grinding aids, lubricants, surfactants, polymers and antistatic agents may be used to prevent agglomeration of the particles and, thus, improve the grinding efficiency. These additives can be in the form of a liquid, a solid, a semisolid, a waxy substance, flakes and micronized beads. A great variety of substances can be used to enhance the milling process. A careful selection with regard to the impact on the quality of the final product as well as environmental issues must be considered. These additives may be selected from the group consisting of ethoxylated alkyl phenol (such as Dodoxynol-5, 6, 7, 9, 12 and Nonoxynol-9, 30), fatty alcohol ethoxylated (such as emulan OG or emulan TO 40), sodium dodecylbenzenesulphonate, sodium dodecyl sulphate, maltodextrin, lecithin (such as phosphatidylcholine, hydroxylated lecithines), fatty acids (such as stearic acid, oleic acid), polysorbates and similar. These additives may be used in a concentration between 0.01 and 10 wt % based on the total weight of the reactive mixture.

Depending on the reactivity of the milled materials and the intensity of the milling process, the milling time can vary between 1 minute and 1 hour. However, the milling time depends of the requisites of the desired product, and it is known that the ball milling needs more milling time for the generation of the desired product.

A mechanical grinding device which controls parameters such as mortar and pestle speed is more appropriate for the synthesis due to the ease of standardization and reproduction of results. For small quantities or at laboratory scale, a micro mill in which the speed of both mortar and pestle can be controlled is preferred. In addition, the mortar and pestle could be covered with a transparent cover for safety. A simple hand mortar and pestle, preferably from agate to avoid contamination, is sufficient for small quantities of sample.

The process of the present invention enables the preparation of metal macrocyclic complexes and/or metal isoprenoid/terpenoid complexes and/or metal linear tetrapyrrole complexes with high yields and under ecofriendly conditions as well as the immediate synthesis of a ready sample for further analysis such as powder X-ray diffraction (XRD) studies and colorimetric measurements. The process of the present invention obviates the troublesome processes to purify the product in solution-based methods or in mechanochemical methods using large amounts of additives. The metal complex in powder form is ready to be characterized by several analytical techniques such as XRD or spectrophotometric measurements, colorimetric measurements.

For XRD studies of the metal complexes of the present invention, a drying process may optionally be carried out at the boiling point of the parental alcohol or at reduced pressure, among others. In this way, the water or parental alcohol, that might be produced in the reaction is eliminated. However, the direct characterization of the metal complex after the mechanochemical reaction without removal of the volatile by-products or without dissolution or recrystallization in other solvents is preferred.

In fact, the product of the present invention, the metal macrocyclic/isoprenoid/linear tetrapyrrole complex in powder form, can be crystalline, semicrystalline or amorphous depending on the selection of the reactants and the molar ratio of the reactants used. If two different metal macrocyclic/isoprenoid/linear tetrapyrrole complexes—with the same or different metal atom—obtained separately by mechanochemistry and by redistribution reaction, are allowed to react by mechanochemistry, cocrystalline complexes can be obtained.

For further applications of the metal macrocyclic/isoprenoid/linear tetrapyrrole complex produced in this invention, such as precursors for film formation techniques or metal oxides with a treated surface such as those to be used in sunscreen or ceramic production, partial or total elimination of the organic material can be conducted. Since the final metal macrocyclic/isoprenoid/linear tetrapyrrole complexes contain mainly carbon, oxygen, nitrogen and hydrogen and the metal or mixture of metals (such as those from phthalocyanine and metal alkoxide), the calcination or pyrolysis of the organic material proceed straightforward. The temperature used for the decomposition of the product prepared by the mechanochemistry method is preferably above 300° C. for three hours, more preferably about 600° C. for three hours, and most preferably about 900° C. for 3 hours. Depending on the film deposition technique to be used, subsequent thermal treatments and dwell times can be optionally adjusted. Thus, if more crystallinity is desired or required, the final metal macrocyclic/carotenoid complexes can be sintered. The calcination process may improve the pigmentary properties such as the color, the texture, weather stability, light fastness and thermal stability of the pigment.

Use of the Metal Complexes for Sunscreens with Concealer Effect

Many people are reluctant to use sunscreens that leave a whitish color or chalky look on the skin. Most natural sunscreens in the market have this disadvantage. The problem is compounded if high amounts of pigments or dyes are added to the sunscreen (to produce the so-called foundation), since it often generates aggregation or irregularities in the sunscreen itself or in the skin and the masking effect is lost. Thus, the desired final naturally looking texture of the sunscreen is not achieved.

Isoprenoid such as carotenoids have been used as skin protectant, skin conditioning and colorants in cosmetic formulations.

One of the objects of the present invention is to use the metal macrocyclic/isoprenoid/linear tetrapyrrole complexes, in particular metal phthalocyanine and/or porphyrin and/or calixarene and/or β-carotene and/or squalene and/or bilirubin complexes, having metals such as titanium, zinc, cerium, iron, aluminium, zirconium, silicon, or germanium and mixtures thereof as a sunscreen. The use of this product as a sunscreen is characterized by a beautiful palette of coloration that covers the whole color spectrum (or their shades) that is distributed homogeneously on the skin. In addition, the use of the metal macrocyclic/isoprenoid/linear tetrapyrrole complexes of the present invention as a sunscreen shows excellent stability in the vehicle without forming precipitates or aggregates in an homogeneous composition. With little changes in the composition of the reactants of the complexes and in the conditions of the reaction and the vehicle of the formulation used, a wide range of skin tones are achieved in line with guides used in cosmetics to match the color of the skin.

The product of this invention to be used as a sunscreen is further formed by the mechanochemical reaction of macrocycles and/or isoprenoids and/or linear tetrapyrroles (e.g. free phthalocyanine/copper phthalocyanine, meso-tetraphenylporphine, 4 tert-butylcalix[4]arene, (3-carotene, squalene, bilirubin) and metal alkoxide. Optionally, between 0.01 and 50 wt % of the mixture of the macrocycle and/or isoprenoid and/or linear tetrapyrrole and metal alkoxide of an additive such as aminoacid (e.g. glycine), peptides (e.g. glicyl-glycin), proteins (e.g. zein, keratin, collagen), synthetic or natural monomer and polymers (e.g. maleic anhydride or lignin), perfume (e.g. pentane-2,3-dione), solvent (e.g. pentane-2,4-dione), acids (e.g. ascorbic acid, tartaric acid, citric acid, acetic acid), fatty alcohol (e.g. cetyl alcohol), natural oils or extracts (e.g. plukenetia volubilis seed) can be added to the prior mixture to impart different colors to the final product. The additive or mixtures of additives can be added conveniently to the metal alkoxide or to the macrocycles and/or the isoprenoids and/or linear tetrapyrroles. Additives should preferentially be inert but it is not restrictive. A controlled reaction with the additives may be advantageous to generate other properties, if desired.

The metal macrocycle/isoprenoid/linear tetrapyrrole complexes having the additive are then mixed with a vehicle or carrier to produce a sunscreen, a foundation, or a hair dye, films, or other materials or formulations. The vehicle or carrier can be any appropriate material such as any base cream, oil, gel or powder.

The vehicle substance or base can be selected from:
a cream base for preparing skin or hair creams without active ingredients, without perfumes, without parabens such as cetomacrogol creme commercially available, comprising water, decyl oleate, cetaryl alcohol, cetearth-20, sorbitol, sorbic acid, or any cetomacrogol creme;
a pasty fatty substance such as waxes, gum or mixtures thereof; an oil, a fatty alcohol, a surfactant, a gel, a powder, a UV filter and/or UV absorber, a skin or hair protecting, an emollient, an humectant, an emulsifier, a skin or hair conditioning, a refatting agent, a masking agent, an emulsion stabilizer, a cleansing agent, an antioxidant, an opacifying agent, a solvent, a viscosity controller, a bulking agent, an abrasive, an anticaking agent, a preservative, a perfume, a buffer agent, an antimicrobial, a salt, water or mixtures thereof or any other cosmetically or pharmaceutically acceptable vehicle.

In addition, the desired sun protection factor can be optionally adjusted by the addition of further synthetic or natural filters such as those listed in the EU Cosmetic Ingredient Database (e.g. titanium oxide).

The weight ratio of the metal complex to the vehicle can be between 1:1000 to 1000:1. Preferably, weight percentage of the metal complex is a range between 0.01 to 30 wt % depending of the solar protection factor that is desired.

The active ingredient for the sunscreen is the titanium complex, or zinc complex or zinc-titanium complex or mixtures thereof. Optionally, another metal or mixture of metals can be used.

This invention covers a mechanochemical process for the synthesis of metal complexes from a macrocycles and/or isoprenoids and/or linear tetrapyrroles and a metal alkoxide. The present invention of metal complexes of macrocycle and/or isoprenoid/linear tetrapyrrole is characterized by a solvent-free synthesis or near solvent-free synthesis with only small amounts of additives. This solvent-free mechanochemical reaction is ecofriendly and avoids the necessity of further steps of solvent evaporation and recycling of the solvent. In addition, the amounts of hazardous by-products diminish. Thus, the entire process for the production of metal complexes of macrocycles and/or metal complexes of isoprenoids and/or metal complexes of linear tetrapyrroles in powder or colloidal form of my present invention fulfills the conditions to be considered green chemistry.

The simplicity, high yield, low cost and ease of scale up makes the process of the present invention to produce metal macrocyclic complexes and metal isoprenoid complexes and metal linear tetrapyrrole complexes and their use as sunscreen with concealer very attractive for industrial applications.

A major advantage of the process of the present invention to produce metal complexes by mechanochemistry over conventional methods to produce organic-inorganic hybrid materials is the rapid completion of the reaction within a few minutes of grinding, the low polydispersity of the finely ground powder and the high stability and homogeneity of the complex obtained. In addition, the process of the present invention provides novel nanostructures similar to those obtained by solution-state methods, for instance after pyrolysis. Since the use of solvent in the present invention is reduced to a minimum or the process is entirely solvent-free, no additional steps are necessary to remove it. In this way, the high yield process of the present invention to produce metal macrocyclic complexes and metal isoprenoids complexes and metal linear tetrapyrrole complexes, in particular, metal phthalocyanines complexes, metal porphyrin complexes, metal calixarene complexes, metal carotenoid complexes and metal squalene complexes is cost-effective.

Another advantage of the present invention is the production of metal complexes with a pleasant odor that does not resemble the original odor of the reactants.

Semipermanent and Temporary Hair Dyes

The urge of solving problems regarding the use of fewer amounts of primary intermediates and couplers without detriment to the performance of hair dye with healthier properties such as protecting the hair from the sun and balancing durability and safety of the dye should be the goal of hair dyeing production.

Natural colorants with synthetic procedures to boost in the lab the already known properties of natural constituents or to find new products or processes thereof has been my goal in the present aspect of my invention on hair dyes and other cosmetic products.

With slight deviations of the formula, the composition of the present invention can be oxidative or non-oxidative. The total or partial diminution of potential allergens or carcinogens such as those containing amine, e.g. p-phenylenediamine, is achieved without detriment to the desired final color.

Hair dyeing compositions containing these new complexes and methods of application are also disclosed: cosmetic dyeing compositions containing, in an appropriate medium and by functionalization of the surface, a complex formed by the reaction of a macrocycle and/or an isoprenoid with a metal alkoxide.

The dyeing formulation of the present invention containing macrocycles and/or isoprenoids and/or linear tetrapyrroles and a metal alkoxide as active ingredient forms a stable color without the use of amines or sulfur containing compounds or salts or oxides commonly used in oxidative hair dyeing. In addition, these compounds can be used, if desired, to add additional properties to the final product.

The dyeing product and dyeing process disclosed in the present invention covers all types of hair and hair colors such as straight, wavy, curly, brown, blond, black, gray or damaged hair. In particular, it covers curly and gray hair, well-known to be very challenging to be colored. Curly and gray hair is covered with an environmental-friendly product and process yielding a durable and stable colored product. Optionally, the hair to be dyed may be also subjected to bleaching prior to the process of the present invention being applied.

The cosmetic dyeing composition of the present invention provides a strong wash fastness, and water repellency that makes the hair dye stable to weather or sporting such as surfing, swimming with resistance to washing out in salty or chlorinated water without detriment to the stability or colored dripping.

The process of the present invention can be used for all kinds of keratinous materials such as eyelashes, eyebrows, skin, nails and tongue.

In the case of free phthalocyanine and/or copper phthalocyanine and/or carotene and metal alkoxide, the color is only formed by those few reactants. There are no sulfur-containing compounds, no salts, no mordants present in the present dye composition to produce color. However, these non-essential compounds may be used if desired. The composition of dyeing hair further consists of at least one coupler and at least one developer conventionally used in oxidative hair dyeing.

Optionally, a coupler or a developer is appropriately mixed either with the macrocycle, carotenoid or the metal alkoxide.

Hence, an oxidizing agent may be also used. Moreover, combining at least one basing agent with at least one oxidizing agent, as in the standard practice, is possible, if desired.

Process for Dyeing in One Step

One aspect of the present invention is a process of dyeing keratinous material by one step for instance by applying to the keratinous materials one of more cosmetic formulations containing the prepared product. Several products of the present invention may be applied together or separately.

Additives can be added before, during or at the end of the hair dyeing.

At least one complex formed in the present invention can be applied directly to the hair in wet (after the reactants are mixed and before the final powder is formed) or dried form (powdered after mechanochemical synthesis with or without additives) or by using a suitable solvent, once the complex is formed with or without surface functionalization. Optionally, the product of the present invention may be functionalized before being applied to the hair in several forms such as paste, cream or colloids or the like, as disclosed for the sunscreen formulation. Optionally the product of the present invention can be applied using an appropriate functionalization of the surface and a suitable solvent.

The present invention also provides a dyeing composition containing the hair dye comprising the complex produced by the reaction of at least one macrocycle and/or at least one isoprenoid and/or at least one linear tetrapyrrole with at least one metal alkoxide, at least one additive selected from the group consisting of a wetting agent, a swelling agent, a penetrant, a pH regulator, a surfactant, a perfume, a synthetic or natural colorant, a thickener, water, etc.

Process for Dyeing in More than One Step, in Loco Complexation Hair Dyeing (ILCHD)

During the process called hereinafter in loco complexation hair dyeing (ILCHD) a macrocycle and/or an isoprenoid and/or a linear tetrapyrrole is applied or put in contact with the substrate such as keratinous material to allow penetration into the cortex of the hair. Once in the fiber, a metal alkoxide is added to the substrate. A complexation is clearly taken place and proven by the formation of color. Presumably, a small increase of the temperature-since the process is exothermic, at least for the complexation of macrocycles/isoprenoids/linear tetrapyrroles with metal alkoxide-enhances the diffusion into the hair cortex and the complex is formed in loco. Thus, the macrocycle and/or the isoprenoid and/or the linear tetrapyrrole are derivatised or complexed in loco, and it is confined into the hair. Once the complex is entrapped in the hair, it is not removed unless rinsing with shampoo. Rinsing with water does not provoke leaching of the metal complex dye of the present invention.

The macrocycle and/or isoprenoid/terpenoid and/or linear tetrapyrrole is diffused into the hair by the addition of a solvent in sufficient amount to solubilize them (preferably warm water or warm alcohol). After the solvent is evaporated, the metal alkoxide is added. Some solvents or additives are allowed to stay in the keratinous material but preferably in a minimal amount.

Since the complexes formed are UV-VIS absorbing materials, they may be detected instantaneously. However, some gentle massage with the hands or comb, to stimulate a triboeffect as in mechanochemical synthesis, can be advantageous.

The ILCHD process is advantageous in comparison with conventional hair dyeing:
No amine-containing compound or amino-phenol compound apart from the macrocycle, isoprenoid/terpenoid or linear tetrapyrrole is needed to form color (however, their use is not restricted to ILCHD);
The formation of color or change of color is instantaneous, leading to less time-consuming process or shorter leave-on times than in natural or oxidative hair dyeing;
Minimal or no damage to the hair;
Several surprising properties to the hair such as manageability of curly hair and relaxing of the hair;

Combination of different colors and shades can be formed by subtle changes in the macrocycles, the isoprenoids, the linear tetrapyrroles, the radicals of the macrocycles, the radicals of the isoprenoids, the radicals of the linear tetrapyrroles, the radicals of the alkoxide and the metal from the metal alkoxide or the macrocycles (chlorophylls, metal phthalocyanines) used.

Some additives can be added to the reactants to generate diverse colors, shades, textures.

A semipermanent hair dye is achieved that does not wash out with water and lasts for more than six washings with or without shampoo. For the hair dye to be more permanent, conventional hair dye procedures as known since the beginning of the 20^{th} century can be applied.

Due to the use of metals such as titanium or zinc and macrocycles and/or isoprenoids and/or linear tetrapyrroles, the hair is protected against radiation such as ultraviolet light. Additionally, more benefits are added to increase the health of the keratinous material coming from the ingredients themselves or by combination effects such as antioxidant, antimicrobials, anti-dandruff, anti-acne, non-comedogenic properties.

The process of application of the hair dye is gentle and pleasing and can be done easily.

Gray hair is dyed to blue, red, yellow, orange, brown and other colors and shades of the whole color spectrum.

The ILCHD is carried out at room temperature. However, the temperature can be varied to increase or decrease the rate of diffusion of the reactants or the complexes formed.

Optionally, as in oxidative hair dye, a pH modifier and an oxidizer can be used to increase or decrease the rate of diffusion of the reactants or the complexes formed.

For the color to develop, there is no need to include amine-containing compounds in the formulation other than the macrocycle/isoprenoids/linear tetrapyrroles used. However, they may be added in the macrocycle compound or in the alkyl or aryl moieties of the metal alkoxides. Moreover, conventional base intermediates and couplers can be used as conventional hair dyeing strategies, and the product of the present invention can even be blended with a conventional hair dye or hair dyeing process to produce a permanent dye.

The compound produced in the present invention is a novel substance, first synthesized by the inventor, with several advantages and with several fields of application.

In the present invention, the color in the complex is determined by the initial compounds, a macrocycle and/or isoprenoid and/or linear tetrapyrrole and metal alkoxide. A great variety of colors are produced without the use of primary intermediaries or couplers such as amine derivatives, peroxides or ammonia. Thus, the hair dye containing a least one macrocycle and/or at least one isoprenoid and/or at least one linear tetrapyrrole may be safer and healthier for the user. Since the amount of ingredients present in the present invention is small, the reproducibility of the final color is higher and the risk of having collateral reactions that can cause damage to the hair and produce toxic intermediates and by-products is minimal. The use of a hair dye with fewer ingredients such as those based on the present invention helps to reduce damage to the hair.

Process for the Formation of Superhydrophobic Surfaces and Superoleophilic Materials

One embodiment of the present invention illustrates the way of forming ultrahydrophobic or superhydrophobic surfaces by using the mechanochemical process to prepare metal complexes of macrocycles/isoprenoids/linear tetrapyrroles by manual grinding using a hand mortar and pestle.

The new method of superhydrophobising materials (e.g. construction material) uses the process to prepare the metal complexes of the present invention. When calixarene is used as a macrocycle and titanium butoxide as a metal alkoxide, a yellow colored complex is obtained that can make a surface to an ultrahydrophobic one and render superamphiphilic properties to the metal macrocycle complex formed in a water compartment. A minimal amount of metal complexes of macrocycles/isoprenoids/linear tetrapyrroles of the present invention forms a self-assembly layer in the water volume/compartment that is deliberately verted into the substract (e.g. porcelain surface from the mortar).

An insulating/superhydrophobic film is formed by the mechanochemical reaction of a metal alkoxide with a macrocycle and/or isoprenoid and/or linear tetrapyrrole in a support or substrate such as porcelain. The unglazed or rough porcelain becomes superhydrophobic in the course of the reaction. Presumably, the metal macrocycle/isoprenoids/linear tetrapyrrole complex of the present invention has performed a self-assembly into the porcelain that was favored by the tribomechanic effect.

Only a simple support but not a sandwich support is used which facilitate the further applications of these hydrophobic surfaces. Only calixarene, a macrocycle without nitrogen, sulfur or fluorine in its structure is used for the formation of the superhydrophobic arrangement. However, the superhydrophobization of the surface can be done with other macrocycles (e.g. porphyrins), isoprenoids and/or linear tetrapyrroles.

When water is added to a porcelain mortar having a superhydrophobic surface with a small amount of powdered metal complexes of the present invention, a spontaneous self-assembly is formed, where the water compartment is surrounded by a layer of metal complexes, wherein presumably the hydrophilic structure is oriented toward the water volume and the hydrophobic part is at the air interface. The compartment/system can be seen as amphiphilic “levitation.” The water evaporation is hindered, since the same amount of water in the same container without amphiphilic “levitation” evaporates quickly. Eventually a double layer of film containing the metal macrocycle/isoprenoid/linear tetrapyrrole complexes is obtained. The slow evaporation allows the formation of the film as the metal organic complex structure is stoked together. The film surrounding the water compartment was acting as a porous membrane letting the water slowly evaporate. The wettability of these membranes can be manipulated by changing, for instance, the structure of the macrocycle and/or isoprenoids and/or linear tetrapyrroles, the metal alkoxide, the molar ratio of the reactants, among others.

The water compartment protected by the self-assembly arrangement of the metal calixarene complex of the present invention can be replaced by a water-soluble compound or substances such an aqueous superparamagnetic fluid or aqueous functionalized magnetic beads, and then the compartment can be displaced by using the superhydrophobic surface of the present invention and a magnetic field. By this way, the superparamagnetic/paramagnetic/magnetic fluid inside the compartment can be seen as a train levitating in the superhydrophobic pathway.

If the water is replaced by RNA, the compartment can be seen as a giant tobacco mosaic virus, where the metal calixarene complexes are the protein containing capsomeres responsible for its motility.

The process for the production of the superhydrophobic surface and the self-assembled membrane comprising the metal complexes of the present invention are low cost, easy to handle, non-hazardous (e.g. elimination of the use of organic solvents and fluorinated/thiosulfate compounds) with a wide range of applications. These metal macrocycle/isoprenoid/linear tetrapyrrole complexes can be used for the self-assembly in the presence of compounds such as proteins/RNA/DNA to simulate the spontaneous formation of capsid or rod-like compartments consisting of layers of the metal macrocycle/isoprenoid/linear tetrapyrrole complexes as a wall and the material to be encapsulated as a reservoir.

The metal complexes of the present invention with enhanced superamphiphilic or superamphiphobic or superhydrophobic/superlipophilic properties may be used as:
Artificial viruses-based materials
Controlled drug delivery
Self-healing artificial skin
Functional membranes
Superhydrophobic paintings or coatings

Process for the Formation of Metal Polymer Nanocomposites

By solvent-free complexation of natural extract of blue-green Spirulina comprising at least one macrocycle—chlorophyll—and at least one isoprenoid/terpenoid—β-carotene—and at least one linear tetrapyrrole—phycocyanin—with a metal alkoxide—titanium(V) n-butoxide—a stable yellow-green colored material in powder form is obtained. When this colored complex is dispersed in water a beautiful brilliant blue coloration is obtained that looks like the Spirulina dispersion in water as supernatant. However, the titanium Spirulina complex of the present invention continues stable for months, while the commercially obtained Spirulina decolorates or loss the blue color after 5 days.

The Spirulina extract comprises also proteins and carbohydrates. Presumably, the complexation of the present invention by mechanochemistry protects the blue pigment phycocyanin by chemical crosslinking with titanium/oxo-titanium bridges and/or the other ingredients such as proteins, chlorophylls and carotenes.

Thus, in a speculative way, the metal alkoxide can be assumed to act as UV absorber or UV protector that ensures the prolonged stabilization of the phycocyanin after complexation. The process is irreversible at least in aqueous conditions.

Since the mechanochemical process was performed at up to 70° C., the metal spirulina complexes of the present invention can be used or further processed at higher temperatures than those recommended by the provider (see Linablue®)

If an ester monomer, e.g. maleic anhydride or a natural polymer e.g. lignin is added the complexation of the present invention by mechanochemical reaction with metal alkoxide generate other kind of materials that are not water soluble.

If Blue Spirulina is used instead of green Spirulina which contains besides phycocyanin, trehalose—a nonreducing disaccharide monomer added for the stabilization of the phycocyanin—, another kind of stable colored material is obtained.

Other uses of the metal complexes of macrocycles and/or isoprenoids and/or linear tetrapyrroles
The present invention, comprising the synthesis of a metal complex in any form (e.g. powder or colloidal) to be used as a catalyst for several reactions or polymerization, is characterized by a one-step production of an ecofriendly catalyst for reactions of polymerization. Transition metal macrocycles may be used to substitute the toxic mercury and tin catalysts for the polyurethane formation, such as titanium or copper.
Use of the metal complexes in all types of catalysis (e.g. use of spirulina metal complexes as a catalyst for the production of biofuels from algae/microalgae.
Several uses in decorative skin design used in different cultural traditions around the world, such as mehndi or sindoor.
Oxidative hair dyeing.
By using lanthanide complexes, a broad variety of materials is obtained that can be applied in magnetic resonance imaging.
Precursor for superconducting.
Chelating agent or intermediate for other reactions either as an intermediate material or finished product from the mechanochemical reaction.
Dye or stain for several substrates such as paper, textile, leather, stone, wood, natural or synthetic polymers or fibers.
Applications related with the optical properties in solar cells, nonlinear optics, display devices, optical data storage.
Medicinal and biomedical applications including both imaging and therapy.
Bactericida, fungicida, as antibiotics.
Edible coating for the conservation of plants, fruits and vegetables.
Skin tissue engineering and wound healing.
Anticorrosion and antifouling.
Drug delivery.
Since algae, in particular cyanobacteria are gram-negative bacteria that obtain their energy through photosynthesis and since they are very resistant to adverse or hostile environments, the black metal complexes obtained from the present invention can be provided as a nutrient or matrix or coverture to cyanobacteria in order to enhance energy production from the photosynthesis. By this way, the production of biomolecules inside the in vitro or in vivo biomass could be more efficient, since they will be protected from the sun (e.g. titanium or zinc black complex) and they can be induced to absorb the light through the whole visible spectrum. Bigger amounts of their biomolecules, such as phycocyanin, carotenes and chlorophylls and others could be obtained. Even fuel can be produced. Their energy can be recollected from the pond as a giant solar cell. Even more, the algae/microalgae could be induced to produce the same kind of black powder.

### EXAMPLES

### Example 1: Niobium-Calixarene Complexes

Materials and Methods
4-Tert-butylcalix[4]arene, 99% (Acros Organics)
Niobium n-butoxide, 99% (metals basis) (Alfa Aesar)
were used as received.

Visual testing was done without or with Pantone Process Color Simulator 1000 guide (solid to process chips) by two observers.

Synthesis

A ceramic mortar was charged with 4-tert-butylcalix[4]arene (0.16 g, 0.25 mmol) and niobium n-butoxide (0.26 g, 0.56 mmol). Immediately the reaction mixture was ground using a hand pestle and the mortar was kept on a heating plate at 40° C. The reaction started with a gray sticky paste that turned, after three minutes of grinding, into a homogeneous beige egg-shell colored powder. The complete yield is only diminished by the difficulty to remove the sticky material from the mortar.

Results and Discussion

Another kind of colored metal complex was formed using niobium as a source of metal from the metal alkoxide and 4-tert-butylcalix[4]arene as a macrocycle ligand. The color matched the Pantone 1345 C or Pantone 155 C solid color standards according to two observers.

The superconductivity and hypoallergenic properties of niobium confer to these metal macrocycle complex a variety of applications such as superconducting foils, catalysis (e.g. photocatalysis, catalysis in reactions/polymerizations).

### Example 2: Copper-Titanium Phthalocyanines Complexes

Materials and Methods
Copper phthalocyanine, dye content ca. 95% (Acros Organics)
Titanium(IV) n-butoxide, 99+% (Alfa Aesar)
were used as received.

Visual testing was done without or with Pantone Process Color Simulator 1000 guide (solid to process chips) by two observers.

Synthesis

Copper phthalocyanine (0.57 g, 0.99 mmol) and titanium butoxide (0.69 g, 2.03 mmol) are ground together in a pre-heated mortar at 40° C. until a homogenously powdered colored material was obtained.

Results and Discussion

By the complexation of the blue copper-phthalocyanine pigment (Pantone 287 C) with titanium butoxide, a blue colored (Pantone 288 C) is produced. The color of the complex formed shows a subtle difference with the copper-phthalocyanine as received. However, the metal complex formed presents improved stability to storage in dispersion. In addition, the resulting metal complexes have better stability in dispersions for instance containing natural sunscreens or other additives. A flocculation-stabilized blue copper phthalocyanine complex is achieved by grinding together a copper phthalocyanine and a metal alkoxide without using halogenation (e.g. chlorination and bromination), commonly used in the industry as a technique to stabilize copper phthalocyanines against flocculation as discussed in the present patent. Neither surface additives (e.g. addition of antiflocculation agents) nor functionalization of the pigment surface (e.g. by addition of strong bases and acids) are necessary to add/perform in the metal phthalocyanine (e.g. copper phthalocyanine) in order to get an improvement in its rheologic behavior in dispersion media (i.e. stabilization of a sunscreen composition comprising titanium dioxide and the titanium copper phthalocyanine complexes produced by the method of the present invention). They might be used but are not necessary in order to stabilize the dispersion of the metal complexes formed. The present method is an economic and an environment-friendly route to stabilize copper phthalocyanine against flocculation in dispersions where titanium dioxide is present. The complexation of the metal phthalocyanine with metal alkoxides by using mechanochemistry (grinding) enhance the dispersibility of these complexes in several water-borne or solvent-borne solvents.

### Example 3: Titanium Complexes of 4-Tert-Butylcalix[4]Arene

Materials and Methods
4-Tert-butylcalix[4]arene, 99% (Acros Organics)
Titanium(IV) n-butoxide, 99% (Acros Organics)
Water, extra pure, deionized (Acros Organics)
were used as received.

Laboratory porcelain mortar (JIPO, unglazed grinding surface, standard form) and porcelain pestle (JIPO unglazed head, standard form) were used to grind the ingredients.

Color Measurements

The color properties of the complexes were evaluated by a spectrophotometer Minolta CM-2002 by reflectance with D65 illuminant, 2° standard observer, and a 1 cm diameter aperture. CIELAB color scales were used to quantify the color of the samples: L* (lightness), a* (redness or greenness), b* (yellowness or blueness). The measurements were performed in duplicate and the instrument records the mean of three values. The calibrations were performed in conforming with the zero and white calibration procedures provided by manufacturer before each use.

The powdered samples and the spectrophotometer were kept at room temperature (22° C.) before measurements. 4-Tert-butylcalix[4]arene was ground before color measurement to eliminate problems associated with the differences in surface and particle size and, in addition, the samples were put in a black vessel of 2 mm height and 15 mm diameter and sealed with 3M scotch transparent tape (TapeClear Paklon/polypropylene with acrylic adhesive) to protect the spectrophotometer from contamination. The measurements were done with the spectrophotometer in upward position. Visual testing was done without or with Pantone Process Color Simulator 1000 guide by two observers.

Synthesis

Titanium n-butoxide (0.17 g, 0.5 mmol) was ground together with 4-Tert-butylcalix[4]arene (0.17 g, 0.26 mmol) by using a laboratory porcelain mortar and pestle in a heating mantel at 30° C. The reaction started with a light yellow paste that turned, after five minutes of grinding, into a yellow colored homogeneous powder. The product of the reaction is let to rest for one hour. The complete yield is only diminished by the difficulty to remove the powder from the mortar.

Results and Discussion

TABLE 1
Hunter L*, a* and b* values of the free 4-tert-butylcalix[4]arene and
the titanium calixarene complex after mechanochemical synthesis by grinding

| Sample | Lightness (L*) | a* | b* | Visual testing |
|---|---|---|---|---|
| 4-Tert-butylcalix[4]arene | 93.76 ± 0.54 | 0.05 ± 0.03 | 1.96 ± 0.04 | white |
| (ground) | | | | |
| Titanium complexes of 4- | 82.95 ± 0.00 | 6.68 0.08 | 51.14 ± 0.54 | Pantone 123 C |
| tert-butylcalix[4]arene | | | | or Pantone 1225 C |

In the table, the values of degree of yellowness, b*, varied significantly from 1.96 for the calixarene before the complexation to 51.14 for the final metal complex when the calixarene was subjected to mechanochemical grinding complexation with titanium butoxide. In addition, the fine yellow colored metal complexes are very stable and homogenous.

When 2 ml of water were verted into the mortar in which the reaction was carried out (after transferring the final complex), an ultrahydrophobic effect was observed on the walls where the reaction was performed. Immediately, a water compartment is formed (or a giant droplet) that was surrounded by some residues of the titanium complex of calixarene that remains in the mortar and can be deformed by subtle movement of the mortar or even cut in small pieces and reformed again. Presumably, the titanium complexes form a self-assembly with the subtract/support, the rough surface of the mortar, where the ultralipophilic part of the complex is inside the mortar surface and the ultrahydrophobic part of the complex at the outside of the surface. The free complex that is inside the mortar forms simultaneously a monolayer/layer that covers the entire water compartment. By this way, the evaporation of the water is hindered and, eventually, when the water is evaporated, a double layer of titanium complexes remains in the mortar.

The mechanochemical method of the present invention for the production of metal calixarene complexes produced by a green process a new complex with superamphiphatic properties never reported before. In addition, a superhydrophobic surface is formed by grinding together only a macrocycle and a metal alkoxide without using nitrogen, sulfur or fluoride commonly used to change the surface or interface properties of materials.

### Example 4: Sunscreen with Concealer Effect

Materials and Methods
Squalene, 99+% (Acros Organics)
Titanium(IV) n-butoxide, 99% (Acros Organics)
were used as received.

Visual testing was done without or with Pantone Process Color Simulator 1000 guide (solid to process chips) by two observers. The L*a*b* values were obtained by conversion of the pantone colors from the web.

Synthesis

Titanium n-butoxide (0.3447 g, 1 mmol) was mixed with squalene (0.410 g, 1 mmol) and immediately milled using a hand agate mortar and pestle. The temperature of the reaction was slowly increased to 70° C. and after five minutes of grinding when no further visible change was detected, a greenish yellow compound was obtained from a beige intermediary color.

Results and Discussion

In the beginning of the grinding process, a beige colored material (Pantone 135 C) is formed that changes to yellow in the process. The color of the final stable material is greenish yellow (Pantone 393 C).

The titanium-squalene complex is used as sunscreen with concealer effect forming a tender and natural texture on the skin while masking without pasting. Although redundant, it is not white, therefore, does not have a chalky appearance.

Different colors can be produced by varying the amount of additives during or after the complexation. The sun protection factor can be adjusted either by varying the amount of metal squalene complexes or by the addition of other natural or synthetic filters in the pharmaceutical carrier.

### Example 5: In Loco Complexation Hair Dyeing of Metal-Free Phthalocyanine and Deoxycholic Acid with Titanium Butoxide

Materials and Methods
Phthalocyanine (Alfa Aesar)
Deoxycholic acid, 99% (Alfa Aesar)
Titanium(IV) n-butoxide, 99% (Acros Organics)
Standard shampoo (e.g. organic shampoo)
were used as received.

Gloves

Gray hair tresses with a 80% of gray coverture.

Dry heat steriliser Melag 91 (Note: it can be replaced by a home hair dryer, an air helmet or let air dry naturally).

Visual testing was done without or with Pantone Process Color Simulator 1000 guide (solid to process chips) by two observers. The L*a*b* values were obtained by conversion of the pantone colors from the web.

ILCHD Method

Hair dyeing process is made according to the ILCHD process of the present invention.

Around 1 g of hair tresses were bundled with two nylon bands, weighted and washed with 10% (w/w) of shampoo with enough water to rinse it. After washing, the tresses were dried in Melag 91 at 60-70° C. until complete dried. Nine hair tresses were subjected to the ILCHD method with the amount of the ingredients used in the recipe.

Phthalocyanine (0.06 g, 0.12 mmol) and deoxycholic acid (0.04 g, 0.10 mmol) were ground together using a hand mortar and pestle and, immediately, titanium butoxide (0.7 g, 2.06 mmol) was added and ground together until a homogenous paste were obtained.

A small amount of paste was added to each hair tresses, and circular massage was applied using one hand with the hair tresses as a mortar and the other hand as a pestle. Optionally, a pestle is used to apply the paste onto the hair by mechanochemical motions similar to the mechanochemical tools.

The tresses were then let to dry in the air dryer at 60-70° C. until completely dried (an air helmet can also be used). The excess of dried complexes onto the hair was removed by combing if desired o rinsing with warm water.

Results and Discussion

The ILCHD is performed using the paste formed when the macrocycle reactants (Phthalocyanine and deoxycholic acid) and the metal alkoxide (titanium butoxide) are ground together and before the reaction is completed inside the mortar. The complexation is accomplished in the hair acting as a substrate or support.

The complexation is then finished in or onto the hair tresses by the gentle massage provided by the hands.

The hair tresses containing the dye complexed in/onto might be gently rinsed and the same procedure can be repeated twice or three times if desired.

In the present protocol, the hair tresses were embedded only once with the complexation reaction mixture.

A beautiful green-bluish color (Pantone 3125 C or Pantone 3135 C) was achieved depending on the gray coverture according to the observers.

If the same reaction is performed to the final powdered material in a hand mortar and pestle and not in/onto the hair, a beautiful green-bluish homogenous colored powder (Pantone 315 C) is obtained. The phthalocyanine chromatically changed from purple/reddish blue (Pantone WWOM-C/Pantone 276 C) to greenish-blue.

The chromic effect varies depending on the composition used in the ILCHD as well.

The hair tresses dye lastes for at least 5 washings with water and/or shampoo. Optionally an appropriate shampoo/conditioner containing similar pigments may be used to provide additional chromatic effects.

### Example 6: Dark Brown Titanium Complexes of Bilirubin

Materials and Methods
Bilirubin, p.a. (Carl Roth)
Titanium(IV) butoxide, 99% (Acros Organics)

Set laboratory porcelain mortar (JIPO, unglazed grinding surface, standard form, 25 ml and porcelain pestle (JIPO unglazed head, standard form, 54 mm) were used to grind the ingredients/reactants.

Visual testing was done without or with Pantone Process Color Simulator 1000 guide (solid to process chips) by two observers using a Color Viewing Light Booth 521, PJC/EC with standardized light D65 from Pantone/Just Normlicht.

The conversion from Pantone colors to L*a*b* values was done from the web.

Synthesis

Bilirubin (0.0286 g, 0.0489 mmol) was ground together with titanium (IV) n-butoxide (0.050 g, 0.1469 mmol) at room temperature for a few minutes. While continuously grinding, the temperature was increased to 40° C. by setting the mortar on a heating plate with digital control of the temperature. The grinding mixture changes from a orange tone to a brown tone.

Results and Discussion

The complexes of titanium bilirubin are obtained in as homogenous dark brown powder as shown in The Table 2.

This brown titanium bilirubin complexes are superhydrophobic. They are extremely difficult to wet even when a lab vortex mixer was used. The metal complexes of bilirubin maintain apart from the water. They are in the upper part of the test tube covering the test tube walls. Presumably by maintaining air in the solid-liquid or solid-solid interface they behave as superhydrophobic materials or surfaces.

TABLE 2
Pantone and Hunter L*, a* and b* values of the finished
titanium bilirubin complexes in powder form after
mechanochemical synthesis by grinding

| | | | | | Visual |
|---|---|---|---|---|---|
| Sample | Pantone | Lightness (L*) | a* | b* | testing |
| Titanium bilirubin | 463-C | 37.27 | 14.68 | 29.24 | Nut Brown |
| complex | | | | | |

### Example 7: Titanium Complexes of Spirulina

Materials and Methods
Titanium(IV) n-butoxide, 99+% (Alfa Aesar)
Spirulina (to avoid confusion, green Spirulina) powder commercially obtained from the organic store (organic powder from Spirulina)

Set laboratory porcelain mortar (JIPO, unglazed grinding surface, standard form, 70 ml) and porcelain pestle (JIPO unglazed head, standard form, 115 mm) were used to grind the ingredients/reactants.

Visual testing was done without or with Pantone Process Color Simulator 1000 guide (solid to process chips) by two observers using a Color Viewing Light Booth 521, PJC/EC with standardized light D65 from Pantone/Just Normlicht.

The conversion from Pantone colors to L*a*b* values was done from the web.

Synthesis Green Spirulina powder (1.00 g) was ground together with titanium(IV) n-butoxide (5.00 g) at room temperature for a few minutes. While continuously grinding, the temperature was increased to 70° C. by setting the mortar on a heating plate with digital control of the temperature. The grinding mixture goes from a green tone to a yellow green tone.

Results and Discussion

Table 3 shows a strong difference in the color of the metal spirulina complex in comparison with the dried spirulina biomass powder.

The final yellowish green powder is stable against light and storage.

Water was added to the green Spirulina and the titanium-green Spirulina complex in order to test solubility. Both are soluble in water. After 5 days, the spirulina biomass as commercially obtained in water lost the color and becomes spoiled.

Thus, while the blue solution of the green spirulina without complexation is already degraded and loss the color completely, surprisingly, the blue colored solution obtained from the green Spirulina subjected to the mechanochemical metal complexation of the present invention continues stable for months in the closed recipient. The blue brilliant color from the titanium-green spirulina complex is stable without fading for months in the same water or after decantation. Presumably, the metal complexation of the present invention protects the phycocyanin pigment even after the increment in temperature during the synthesis.

The spirulina powder being a biomass from the Arthrospira species comprising macrocycles—Chlorophylls—and isoprenoids—β-carotenes—and linear tetrapyrroles—phycobilinprotein—, the process of the complexation of the present invention can be used to protect the biomass from degradation during the extraction techniques using solvents, e.g. water extraction. Not only the process of producing a stable metal spirulina complex of the present invention can be performed in commercially obtain algae/microalgae biomass but it can also be used before, during or after the extraction processes of this biomass/biocompounds well known in the art. In this embodiment, the solvent-free mechanochemical metal complexation of the biomass was performed without additives.

Additionally, the maceration process well known for destroying cell walls can be accomplished by the addition of metal alkoxides during the mechanochemical processing.

TABLE 3
Pantone and Hunter L*, a* and b* values of the green Spirulina biomass and the final
spirulina complexes in powder form after mechanochemical synthesis by grinding

| Sample | Pantone | Lightness (L*) | a* | b* | Visual testing |
|---|---|---|---|---|---|
| Spirulina biomass powder as | 5467-C | 18.95 | −11.23 | −1.23 | Dark pine |
| commercially obtained before | | | | | green/Swiss pine |
| mecanochemical reaction with | | | | | forest |
| titanium(IV) butoxide | | | | | |
| Final metal complex in powder | 5753-C | 41.8 | −8.77 | 24.59 | Dark emerald |
| form | | | | | green |

The process of the complexation of the present invention protects the microalgae and hinders the quickly degradation of the green Spirulina biomass when is under aqueous conditions. The change in color by metal complexation of the dried biomass but not from the pigments within the biomass (at least visually) it is a relevant characteristic of the present invention—at least the blue color is similar from those obtained from the green spirulina extraction in water. The powders themselves after the finished mechanochemical complexation are ready to be used as powder cosmetic.

Regardless of the kind of extraction process used to produce the spirulina powder, the solvent-free mechanochemical synthesis of the present invention protects the bioalgae as well as their components from quickly degradation.

### Example 8: Tantalum Complexes of Polymer-Blue Spirulina

Materials and Methods
Tantalum (V) ethoxide (Gelest/Mitsubishi)
Blue Spirulina powder (100% natural Arthrospira Platensis extract, powder, 27% Phycocyanine)
Maleic anhydride (≥99.5 Zur Synthese, Karl Roth)

Set laboratory porcelain mortar (JIPO, unglazed grinding surface, standard form, 25 ml) and porcelain pestle (JIPO unglazed head, standard form, 54 mm) were used to grind the ingredients/reactants.

Visual testing was done without or with Pantone Process Color Simulator 1000 guide (solid to process chips) by two observers using a Color Viewing Light Booth 521, PJC/EC with standardized light D65 from Pantone/Just Normlicht.

The L*a*b* values were obtained by conversion of the pantone colors from the web.

Synthesis

Blue Spirulina (0.40 g) was ground together with tantalum(V) ethoxide for 5 min at room temperature. Subsequently maleic anhydride was added while continuously grinding and careful increasing the temperature to 70° C. (a IKA heating plate having a digital temperature control was used to warm the mortar). The reaction mixture changes from pasty at the beginning to liquid in the intermediate stage to the finished sticky paste after approximately 10 min. After further grinding, a homogenous colored powder is obtained. The reaction mixture changes from dark blue to the final light blue.

Results and Discussion

Clearly, the reaction mixture changes from a dark blue to the Pantone 2905-C light blue. The paste can be used for manufacturing articles or devices. Since tantalum is a bioinert metal, Spirulina is a natural and healthy colorant, maleic anhydride is a versatile additive/monomer with a rich chemistry (from the COSING DATABASE), a non-toxic polymer was produced by mechanochemical reaction of Spirulina and tantalum ethoxide without using aqueous or organic solvents and only use a monomer as an additive to favor further processing. Optionally, the grinding mixture can be further ground to a finely divided powder.

The complex could also be used as coating for instance for orthopedic implant material since tantalum is highly bioinert

TABLE 4
Hunter L*, a* and b* values of the final tantalum
polymer spirulina complexes in powder form after
mechanochemical synthesis by grinding

| | | | | | Visual |
|---|---|---|---|---|---|
| Sample | Pantone | Lightness (L*) | a* | b* | testing |
| Tantalum blue- | 2905-C | 77.15 | −13.41 | −23.28 | Marinai |
| Spirulina | | | | | |
| polymer complex | | | | | |

If the maleic anhydride is replaced by other additives such as monomers (e.g. acrylic acid, methacrylic acid, bytyl acrylate, phenol-formaldehyde or styrene), polymers (cellulose, lignin or glycogen), metal complexes are obtained with diverse properties.

### Example 9: Brown Titanium Curcumin-Lignin-Spirulina Complex

Materials and Methods
Curcumin (food additive E 100, kurkum, turmeric yellow CI or natural yellow 3) from the local food store.
Lignin UPM BioPIVA™ 199, kraft lignin powder (UPM, The Biofore Company)
Blue Spirulina powder from the organic store (100% natural Arthrospira Platensis extract, powder, 27% phycocyanin)
Titanium(IV) n-butoxide, 99+% (Alfa Aesar)

Set laboratory porcelain mortar (JIPO, unglazed grinding surface, standard form, 70 ml) and porcelain pestle (JIPO unglazed head, standard form, 115 mm) were used to grind the ingredients/reactants.

Visual testing was done without or with Pantone Process Color Simulator 1000 guide (solid to process chips) by two observers using a Color Viewing Light Booth 521, PJC/EC with standardized light D65 from Pantone/Just Normlicht.

The L*a*b* values were obtained by conversion of the pantone colors from the web.

Synthesis

Curcumin (0.4 g), lignin (0.4 g) and Spirulina (0.83 g) were ground together in a porcelain mortar and pestle until a homogenous physical mixture is obtained. Then, titanium butoxide (2.48 g) was added and the entire mixture is ground. The mortar was put in a heating plate and the temperature was gradually increase to 72° C. The grinding mixture changes from a dark fluid paste to a sticky paste after 15 min of continuously grinding. At this stage the paste can be used for further processing. A brown powder is obtained if the grinding is continued, i.e. the finished metal complex in powder form.

Results and Discussion

The grounded mixture of curcumin, lignin and Blue Spirulina was of a gray green color as shown in table

TABLE 5
Hunter L*, a* and b* values of the curcumin-lignin-blue Spirulina physical
mixture and the final titanium curcumin-lignin-spirulina complexes
in powder form after mechanochemical synthesis by grinding

| Sample | Pantone | Lightness (L*) | a* | b* | Visual testing |
|---|---|---|---|---|---|
| Physical mixture of the | 5477-C | 36.66 | −11.97 | −1.63 | Gray-green |
| macrocycles and isoprenoids | | | | | |
| and linear tetrapyrroles and | | | | | |
| natural polymer lignin as | | | | | |
| additive before reaction with | | | | | |
| titanium(IV) butoxide | | | | | |
| Final metal complex in powder | 4625-C | 22.61 | 15.55 | 17.29 | Dark Brown |
| form | | | | | |

After the mechanochemical reaction using titanium(IV) n-butoxide as metal alkoxide, a beautiful brown is obtained.

It is clear that the process of the present invention is not a mixture of ingredients such as those used to produce color by the painter to achieve a specific tone. To the contrary, it is a mechanochemical reaction where the final color cannot be predicted in advance. Once a color of one particular reaction is known, changes in the proportions of the reactants can generate the desired color.

It might be decided to interrupt the process of the mechanochemical reaction when a sticky paste is formed after some time of mechanochemical grinding or milling, i.e. the intermediate material. At this stage, the reaction mixture can be used for further processing for instance to the manufacture of devices, to produce paints or coatings and so forth.

### Example 10: Black Color by Mechanochemical Metal Complexation of Natural Ingredients

Materials and Methods
Curcumin (food additive E 100, kurkum, turmeric yellow CI or natural yellow 3) from the local food store.
Lignin UPM BioPIVA™ 199, kraft lignin powder (UPM, The Biofore Company)
Blue Spirulina powder commercially available from the organic store
Titanium (IV) n-butoxide, 99+% (Alfa Aesar)
Water, extra pure, deionized (Acros Organics)
Carbon disulfide, 99.9%, for spectroscopy (Acros Organics)
Pyridine, ultrapure, spectrophotometric grade 99.5+% (Acros Organics)
Methyl sulfoxide, extra pure, 99.8+% (DMSO, Acros Organics)
N,N-dimethylformamide, 99.8% ACS reagent (DMF, Acros Organics)

Set laboratory porcelain mortar (JIPO, unglazed grinding surface, standard form, 70 ml) and porcelain pestle (JIPO unglazed head, standard form, 115 mm) were used to grind the ingredients/reactants.

Visual testing was done without or with Pantone Process Color Simulator 1000 guide (solid to process chips) by two observers using a Color Viewing Light Booth 521, PJC/EC with standardized light D65 from Pantone/Just Normlicht. The L*a*b* values were obtained by conversion of the pantone colors from the web.

Synthesis

Lignin (0.1 g), curcumin (0.1 g) and blue Spirulina (0.82 g) are ground together until a homogenous mixture of was obtained. Titanium (IV) n-butoxide (2.0 g) was added, the mortar is put on a heating plate, and the temperature was gradually increased to 65° C. while grinding to a fine divided powder. The final color of the complex obtained resembles the color of the anthracite, it means, a near-black or very dark gray.

Results and Discussion

TABLE 6
Hunter L*, a* and b* values of a curcumin-lignin-blue Spirulina physical
mixture and the final titanium curcumin-lignin-spirulina complexes
in powder form after mechanochemical synthesis by grinding

| Sample | Pantone | Lightness (L*) | a* | b* | Visual testing |
|---|---|---|---|---|---|
| Physical mixture of the macrocycles and | 302-C | 21.51 | −13.48 | −27.98 | Navy blue |
| isoprenoids and linear tetrapyrroles and | | | | | |
| natural polymer lignin as polymer | | | | | |
| additive before reaction with | | | | | |
| titanium(IV) butoxide | | | | | |
| Final metal complex in powder form | 426-C | 15.76 | −0.79 | −2.31 | Anthracite |
| | | | | | black |

The black titanium complex obtained in the present example resembles anthracite powder or hard coal as shown in Table 6.

Carbon disulfide is a non-polar solvent which is commonly used as solvent for metal complexes.

Curcumin is completely soluble in carbon disulfide forming a yellow solution. Lignin is partially soluble in carbon disulfide forming a brown solution, and spirulina can be dispersed in carbon disulfide forming a blue dispersion. The black color pigment/dye obtained by the solvent-free mechanochemical synthesis of curcumin, lignin and spirulina with titanium(IV) n-butoxide obtained with this formulation is completely insoluble in carbon disulfide, and only a black precipitate is formed at the bottom of the test tube. It is clear that all of the reactants reacted by this mechanochemical process. The mechanochemical reaction promotes a chemical change. Hence, the black titanium complex of curcumin and lignin and spirulina is a product of a mechanochemical reaction of the tree compounds with titanium alkoxide and not the simple physical mixture of ingredients.

The black titanium complexes are partially soluble and stable in water forming a blue solution with some red precipitates. If pyridine, DMSO and DMF are used as a solvent, other colored supernatants are formed.

A modified spirulina colorant that continues highly soluble in water and highly stable in water after the mechanochemical treating (grinding or milling) with metal alkoxides but it is converted to a colorant which is insoluble in organic solvents such as carbon disulfide. The mechanochemical metal complexation of the present invention is presumably a method of protection of the phycocyanin/phicobilin dye by the metal alkoxide and the other reactants lignin, a polymer and Curcuma, a source of terpenoids. The phycocyanin structure is composed of two protein subunits, α and β chains and one phycocyanobilin is bound to the α sub unity and two phycocyanobilins are bound to the β subunity via thioether bond. Only by speculation, given the globular structure and the planar framework from the lignin and curcumin, a forced mechanochemical reaction under solvent free condition might be assumed to obligate the lignin/curcumin to protect or cover the phycocyanin protein by the titanium or titanium bridges. The process could be similar to the fusion between lignin, cellulose and hemicellulose in nature. In addition, given the fact that proteins present different conformations at different environments/solvents, some modifications in the structure change also the conformation at different solvents. Nevertheless, the modified macrocycle/isoprenoid/linear tetrapyrrole complex is more stable than the counterparts in the same media. The black metal complexes of the present invention changed the conformation at different conditions.

This process of modification of the dye structure is perhaps more thermodynamically stable in water than the phycocyanine structure without modifications. If water is absent, a black/anthracite color is displayed. In addition, as in the case of other macrocycles/isoprenoids/linear tetrapyrroles such as calixarenes, the Spirulina complexes present superhydrophilic properties.

A very stable black compound in powder form that is obtained by complexation of natural ingredients or waste products from the paper industry and a metal alkoxide such as titanium alkoxide is obtained. Its insolubility in organic solvents like carbon disulfide makes it very attractive to be used as an organic pigment. It is an insoluble organic pigment with partial solubility in aqueous solvents and organic solvents, such as other macrocycles modified by the solvent-free mechanochemical reaction of phthalocyanine/metal phthalocyanines with metal alkoxides. Therefore, these organic pigments can be used in the coloring of inks, paints, rubber products—as filler or reinforcement agents-, plastic products. Some properties such as good dispersion properties or high tinting strength are not there immediately during the synthesis and must be obtained through the so-called pigmentation processes. Those are some of the tasks of the producers of pigments.

Thus, by slight modifications of the proportions of the reactants and the type of reactants themselves, the whole spectrum of colors can be obtained by the solvent-free mechanochemical reaction of at least one macrocycle and/or at least one isoprenoid and at least one linear tetrapyrrole with at least one metal alkoxide. With further processing of the intermediate compounds or the finished complexes, a very wide gamma of new colorants with improved or novel properties can be obtained.

Given the fact that black color are scare in nature and it can mainly be industrially obtained from the fuel combustion such as carbon black, the process of my invention to produce black colors contribute in a ecofriendly way to the production of black colors that could replace those obtained by fuel combustion such as carbon black which is mutagenic and carcinogenic. Moreover, the dyes produced by the process of the present invention produce colors of the whole spectrum by green chemistry.

The photosynthesis use chlorophylls and accessory pigments such as carotenes and phycobilins—the pigments contained in Spirulina—to generate energy and carbohydrates from the light. However, such mixtures of pigments do not absorb the whole visible range. If those photosynthetic pigments were black or near-black, the photosynthesis would be more efficient, since more energy from the sun would be absorbed.

My invention could contribute positively to the industrial production of colorant materials from natural resources and industrially produced metal alkoxide. Thus, it is a semisynthetic and sustainable way to produce novel value-added products.

I could envision that if my black pigments were added to the ponds or lakes where algae/microalgae are growing, they would induce a cost-effective production of biomolecules by those natural resources. By this induced modification by adsorption or absorption, algae/microalgae would take more advantage of the sun light and perhaps their photosynthesis process would be more efficient.

Although the embodiments of the present invention have been described and illustrated in detail, it will be understood that the present invention is not limited to the above-described embodiments, and various modifications in design may be necessary without departing from the spirit and scope of the invention defined in the claims.

### CITATIONS

¹ IUPAC, Compendium of Chemical Technology, 2nd ed. (the “Gold book”). Compiled by A. D, McNaught and A. Wilkinson (1997). Blackwell Scientific Publications, Oxford. Online version 2019 created by S. J. Chalk.
² Mormann W. & Hellwich K.-H. (2008). Structure-based nomenclature for cyclic organic macromolecules. IUPAC recommendations 2008). Pure and Applied Chemistry, 80, 201-232.
³ Bowman-James K. (2006). “Macrocyclic Ligands”, in: Encyclopedia of Inorganic and Bioinorganic Chemistry, John Wiley & Sons.
⁴ Moss G. P. (1987). Nomenclature of Tetrapyrroles, Pure Appl. Chem. 59, 779-832.
⁵ Crichton R. (2019). Biological Inorganic Chemistry, a new introduction to molecular structure and function, Academic Press, Chennai.
⁶ Höcker H. (2009). Cyclic and macrocyclic compounds—a personal review in Honor of Professor Leopold Ružička, Kem. Ind. 58(2), 73-80.
⁷ Afzal A., Oriqat G., Khan M. A., Jose J. and Afzal M. (2013). Chemistry and biochemistry of terpenoids from Curcuma and related species, Journal of biological and active product from nature, 3, 1-55.
⁸ a) Horn, M. & Horns U. (“Alkoxides, metal”, in: Kirk-Ohmer Encyclopedia of Chemical Technology, Vol. 2, 4th ed., 21-29, John Wiley & Sons, New York. b) Bradley, D. C., Mehrotra, R. C. & Gaur, D. P. (1978). Metal Alkoxides, Academic Press, London; c) Bradley, D. C., Mehrotra R. C., Rothwell I. P. and Singh A. (2001) Alkoxo and aryloxo derivatives of metals, Academic Press, London.
⁹ Buchler J. W. (1978). Synthesis and properties of metalloporphyrins, in: The Porphyrins, vol 1. Structure and synthesis. Part 1, 389-483 (Eds Dolphin D), Academic Press, New York.
¹⁰ a) Herbst W. & Hunger K. (2004). Industrial Organic Pigments, production, properties and applications, 3^{th} edition, completely revised edition, John Wiley & sons, Weinheim. or b) Hunger H. & Schmidt M. U. (2018) Industrial Organic Pigments: production, crystal structures and properties, Fourth completed revised edition, John Wiley & Sons, Weinheim.
¹¹ U.S. Pat. No. 5,318,623
¹² JP1994293769
¹³ Ralphs, K., Zhang C. James, S. L. (2017). Solventless mechanochemical metallation of porphyrins, Green Chem, 19, 102-105.
¹⁴ Atoyebi, A. O. and Bruckner C. (2019). Observations on the mechanochemical insertion of zinc(II), copper(II), magnesium(II), and selected other metal(II) ions into porphyrins, Inorg. Chem., 58, 9631-9642.
¹⁵ JP1996311365
¹⁶ Czackler M., Artner C., Maurer C, and Schubert U. (2014). Calix[4]arene derivatives of titanium and zirconium alkoxides, Z. Naturforsch, 69b, 1253-1259.
¹⁷ Neri P., Sessler J. L., Wang M.-X., Eds (2016). Calixarenes and Beyond, Springer, Dordrecht.
¹⁸ La Barre S. & Bates S. S. (2018). Blue Technology, Production and Use of Marine Molecules. Vol. 1 and Vol. 2, Wiley-VCH, Weinheim
¹⁹ https://www.earthrise.com/what-is-linablue;
²⁰ https://www.dic-global.com/en/products/natural_colorants/
²¹ https://exberry.com/nl/nieuws-evenementen/spirulina-breakthrough;
https://exberry.com/newsletter/2020/EXBERRY_Trendreport_BlueCampagne_2020.pdf
²² Erk P. & Hengelsberg H. (2003). “Phthalocyanine Dyes and Pigments”, in: The porphyrin Handbook, vol. 19/applications of phthalocyanines (Eds Kadish K. m., Smith K. M. and Guillard R.), Elsevier Science, USA.
²³ Focsan A. L. et al (2019), Supramolecular carotenoid complexes of enhanced solubility and stability—the way bioavailability improvement, molecules, 24, 3947.
²⁴ WO2015/090697A1
²⁵ FR2929957A1
²⁶ Tobin, D. J (Ed) (2005) Hair in toxicology: an important bio-monitor, The Royal Society of Chemistry publishing, Cambridge.
²⁷ Corbett, J. F. (1971) “Hair Dyes”, Chapter VII, in: The Chemistry of Synthetic Dyes, Vol. 5 (Ed Venkataraman K.) Academic Press, New York and London.
²⁸ WO2006/106366A1
²⁹ Putzig D. E & Moncarz J. R. (2007). “Titanium compounds, Organic”, in: Kirk-Ohmer Encyclopedia of Chemical Technology, Vol. 25, 5th ed., John Wiley & Sons, New York.
³⁰ Pomogalio A. & Kestelman V. (2005) Chapter 4, 5 and 6. In Metallopolymer Nanocomposites, Springer series in material sciences, vol. 81, Springer, Berlin-Heidelberg.
³¹ Dodziuk, H. (2002). Introduction to supramolecular chemistry, Kluwer academic publishers, Dordrecht
³² Davis, F. & Higson S. (2011). Macrocycles: Construction, Chemistry, and Nanotechnology applications, First Edition, John Wiley & Sons, Malaysia.
³³ Turova, N. Y., Turevskaya E. P., Kessler V. G. & Yanovskaya M. I. (2006). The Chemistry of Metal Alkoxides, Springer Science & Business Media, Dordrecht.
³⁴ Tanaka, K. (2009). Solvent-free organic synthesis, Second Ed, completely revisited and updated version, Wiley-VCH Verlag GmbH & Co. KGaA, Weinheim.
³⁵ Hernández J. G. & Bolm C. (2017). Altering product selectivity by mechanochemistry. J. Org. Chem., 82, 4007-4019.

1. Metal complex in any physical form comprising the product of the reaction of
(a) at least one compound selected from macrocycles and/or isoprenoids and/or linear tetrapyrroles and
(b) at least one metal alkoxide obtainable by mechanochemistry (grinding or milling) under solvent-free conditions wherein the mechanochemistry includes the use of a mechanical or hand mortar and pestle, high speed milling, ball milling, attrition milling, planetary milling or extrusion.

2. Metal complex according to claim 1 further comprising
(c) at least one additive.

3. Metal complex according to claim 1 wherein:
(a) the at least one macrocycle compound, either synthetic or of natural origin, containing oxygen, nitrogen, sulfur or phosphorus donors/elements as replacement of other skeletal atoms (e.g. nitrogen or carbon in porphyrins) and other modifications, substituted or unsubstituted, with planar or non-planar structures, with or without containing a complexed metal ion is selected from:
Polyaza macrocycles (simple polyaza macrocycles, cyclidenes, sepulchrates, bis-macrocylces, expanded porphyrins), e.g. cyclam or polyaza criptate;
Simple and multi-ring aromatic compounds, e.g. anullenes, pyrene, coronene, ovalene, perylene, phenanthrene, kekulene, hexahelicene, graphite, graphene or fullerene;
Tetrapyrroles and their relatives (b-substituted porphyrins, meso-substituted porphyrins, metal porphyrins, ring-expanded porphyrins, ring-contracted porphyrins, reduced porphyrins), e.g. porphyrins, meso-tetraphenylporphine; chlorophylls (a, b, c and d), chlorophyllin, bacteriochlorophylls; chlorins; bacteriochlorin; carotenoporphyrins; corrin; corrole; sapphyrin; heme; hemochrome; hemin or hematin;
Fused macrocyclic tetrapyrrole systems, e.g. phthalocyanines or metal phthalocyanines, e.g. copper phthalocyanines, titanyl phthalocyanine, tetrabenzoporphyrin, mono, bis, tris and polyphthalocyanines; polythia, polyphospha or polyarsa macrocycles;
Mixed donor macrocycles, e.g. cryptands, compartmental ligands, catenanes or rotaxanes;
Polyoxa macrocycles or crown ethers, e.g. polyether macrocycles, lariat ethers, spherands or hemispherands;
Calixarenes; pillarenes; resorcinarenes; cavitands; carcerands;
Terpenoid macrocycles, e.g. taxol, rapamycin, ascomycin or tacrolimus;
Alkaloid macrocycles, e.g. Trabectedin;
Macrolactones; macrolides; cardenolides; bufadienolide, e.g. erythromycin;
Peptide or protein-based macrocycles (globin), e.g. hemoglobin, myoglobin, picket-fence porphyrin complex, or hemeprotein;
Fullerene macrocycles and their related materials, e.g. endohedral or exohedral fullerenes, graphenes, graphite, or carbon nanotubes;
Organic zeolites;
Dendrimers;
Polyketide macrocycles or macrolides;
Perylene-based macrocycles:
Cyclophanes, e.g. paracyclophanes;
Cyclotetraicosaphenylene;
Cyclodextrins;
Cucurbiturils;
Vitamins and derivatives, e.g. vitamin B (or cobolamin); • Macrocyclic bile acid, e.g. cholic acid, chenodeoxycholic acid, deoxycholic acid and lithocholic acid;
Other naturally occurring macrocycles displaying a variety of biological activities (immunosuppressant, antibiotic, anticancer, antifugal, ACE inhibitor), e.g. FK-506, tetracycline, aminoglycoside streptomycin, paromomycin, vancomycin, epothilone B, geldanamycin gentamicin, K-13, amphothericin B, amoxicillin or clarithromycin;
Formulations or compounds containing macrocycles including nanoparticles, liposomal encapsulation, phospholipid complexes, emulsions, capsules, tablets and powders, either used alone or in combination with other compounds, such as medicines, antibiotics, polyphenols, alkaloids (piperine), carbohydrates (monosaccharides, oligosaccharides and polysaccharides), aminoacids, peptides, proteins; herbal preparations, such as extracts or tinctures containing said macrocycles;
Isoprenoid/terpcnoid-modified macrocycles, e.g. cytoporphyrin;
Mixtures or modifications thereof,
(b) the at least one isoprenoid, either synthetic or of natural origin, containing oxygen, nitrogen, sulfur, fluorine or phosphorus donors/elements as replacement of other skeletal atoms (e.g. fluorine in retinoids) and other modifications, substituted or unsubstituted, with or without containing a complexed metal ion, is selected from:
Carotenoids either carotenes or xanthophylls (hydrocarbons, alcohols, glycosides, ethers, epoxides, aldehydes, acid and acid esters, ketones, esters of alcohols, apo-carotenoids, nor- and seco carotenoids, retro-carotenoids and retro-apo-carotenoids), e.g. acyclic carotenes, lycopene, carotenes (s, y, b, e, g, k, f, X), capsanthin, lutein, criptoxanthin, zeaxanthin, neoxanthin, violaxanthin, flavoxanthin, astaxanthin, bixin, crocetin, crocin, fucoxanthin or iridoids;
Terpenoids (hemiterpenoids, monoterpenoids, sequiterpenoids, diterpenoids, sesterterpenoids, triterpenoids, tetraterpenoids, polyterpenoids), e.g. isoprene; prenols; dolichol; polyprenols; steroids; sterols/phytosterols, e.g stigmaterol; carotenoids; ginkgolide; bilobalide; citral; menthol; camphor; salvinorin A; cannabinoids; farnesol; carvone; eucalyptol or squalene;
Macrocycle-modified isoprenoids/terpenoids, e.g. carotenoporphyrins or carotenofullerenes;
Cyclic monoterpenoids (iridoids), e.g. genipin, geniposide, aucubin or catalpol;
Retinoids (retinol, retinal, retinoic acid, retinyl esters, nor- and seco retinoids, retro-retinoids), e.g. vitamin A;
Vitamins and derivatives, e.g. vitamin K;
Tocopherols, e.g. a-tocopherols or vitamin E;
Peptide or protein-based isoprenoids/terpenoids, e.g. prenylated proteins;
Formulations or compounds containing isoprenoids/terpenoids including nanoparticles, liposomal encapsulation, phospholipid complexes, emulsions, capsules, tablets and powders, either used alone or in combination with other compounds, such as medicines, antibiotics, polyphenols, alkaloids (piperine), carbohydrates (monosaccharides, oligosaccharides and polysaccharides), aminoacids, peptides, proteins; herbal preparations, such as extracts or tinctures containing the isoprenoids/terpenoids, e.g. paprika oleoresin;
Mixtures or modifications thereof,
(c) the at least one linear tetrapyrrole, either synthetic or of natural origin containing oxygen, nitrogen, sulfur, fluorine or phosphorus elements as replacement of other skeletal atoms (e.g. oxygen in phycoerythrobilin) and other modifications, substituted or unsubstituted, with or without containing a complexed metal ion is selected from,
linear tetrapyrroles (bilanes, bilins, bilenes, biladienes), e.g. biliverdins, mesobiliverdins, bilirubins, mesobilirubins, urobilins, stercobilins, urobilinogens, phycoerythrobilin, phycocyanobillin/phycobiliverdin, phycoviolobilin, secocorrin or phycourobilin: • Phycobilins, e.g. phycoerythrobilin, phycocyanobillin, phycoviolobilin or phycourobilin;
Protein-pigment complexes, e.g. phycocyanin, phycoerythrin phycoerithrocyanin, allophycocyanin or phytochromobilin;
Relatives to linear tetrapyrrole compounds, e.g. linear tripyrroles (e.g. tripyrrin or reduced tripyrrin); dipyrroles, (e.g. dipyrrin (formerly dipyrromethene), -dipyrrinl-(10H)-one (formerly pyrromethenone) or dipyrromethane (formerly dipyrrylmethane),
Mixtures or modifications thereof,
(d) The at least one macrocyle and/or the at least one isoprenoid and/or the at least one linear tetrapyrrole as extracts in any form, tinctures, essential oils or powders/biomass (from each part of the natural source or the whole source) either synthetic or of the natural origen, either with natural or delivered induced modification, either natural or synthetic product after recombinant techniques or physicochemical techniques, is selected from: Terpenoids, e.g. from turmeric, ginger or rubber tree;
Cannabinoids, e.g. myrcene, β-cariophyilene or limonene;
Indoids (from Gentiannceae, Rubiacene, Ericaceae, Valarianaceae, e.g. genipin;
Coenzymes, e.g. ubiquinone(Coenzyme Q₁₀):
Protein-based macrocycles/isoprenoids linear tetrapyrroles, e.g. phycocyanin or hemoglobin;
Plants of genera: Hevea, Landophia, Taraxacum, Palaquium, Amaranthus, Zingiber, Vitis, Citrullus, Citrus, Coriandrum, Cotinus, Euphrasia, Lavandula, Verbenacea, Illicium Carum, Mentha, Calendula, Bursera, Artemisia, Vachellia, Cinnamomum, Eucalyptus, Glycyrrhiza/liquorice Syzygium, Betula, Backhousia, Leptospermum, Ocimum, Solanum, Helianthus, Cannabis, Lupinus, Brassica, Crataegus, Curcuma, Gardenia, Crocus Lawsonia, Indigofera, Genipa, Oenothera, Lespedeza, Passiflora, Hamamelis, Theobroma, Coffea, Chamaemelum, Quercus, Capsicum, Molva, Bixa, Tagetes, Cynara, Glycine, Asperula, Angelica, Hieracium, Ammi, Melilotus, Aesculus, Lithospermum, Solidago, Origanum, Camellia, Schisandra, Hibiscus, Rosa, Ribes, Acacia, Bactris, Rhus, Gingko, Juglans, Moringa, Lavandula or Persia;
Algae (brown algae (e.g. kelp), red algae (e.g. Gracilaria, Porphyra), green algae (e.g. Haematococcus pluvialis, B. braunii), e.g. astaxanthin;
Microalgae (cyanobacteria and eukaryotic algae), e.g. Arthrospira/Spirulina (e.g. Arthrospira platensis, A. fusiformis, A. maxima), Chlorella (e.g. vulgaris, pyrenoidosa), Dunadiella salina, Aphanizomenon Flos-aqua), e.g. blue Spirulina extract, Spirulina biomass powder, Chlorella biomass/powder, asthaxanthin powder, β-carotene or chlorophylls;
Fungi (e.g Aspergillus, Trichoderma, Penicillium, Bipolaris), e.g. antibiotics, Phytohormones, metacridamides, trichothecenes or macrocytic polylactone;
Bacteria (from E coli, Streptomyces Hygroscopicus), e.g. aromadendrene, geldanamycin;
Yeast (genetically modified), e.g. farnesene;
Animals or humans (e.g. sterols or steroid hormones, pheromones (e.g. dendrolasin, iridomyrmicin), squalene, lanosterol, cholesterol), e.g. Euphausia pacifica from krill, Euphasia superba from krill or Pandalus borealis from shrimp;
Mixtures or modifications thereof.

4. Metal complex according to claim 1 wherein the at least one metal alkoxide (b) is a compound of formula M(OR)x or [M(ORx)] or heterometallic alkoxide such as mixed halide-alkoxides or a bimetallic alkoxide (double alkoxide), or a polymeric metal alkoxide or metal-oxo-alkoxides or metal aryloxide wherein,
(a) M is one or more elements from the elemental periodic table, preferably titanium, zirconium, hafnium, vanadium, aluminium, germanium, silicon, niobium, lithium, tantalum, zinc, magnesium, antimony, indium, gallium, copper, holmium, tin, lanthanum, erbium, terbium, barium, gadolinium, yttrium, tantalum, dysprosium, cobalt, tellurium, lead, bismuth, calcium, cerium, iron, strontium, molybdenum, tungsten, neodymium, nickel, samarium, europium, osmium, praseodymium, boron, sodium, potassium, thallium, scandium, chromium, manganese, platinum, ruthenium, gold, silver, beryllium, cadmium, mercury, thorium, selenium, or mixtures thereof,
(b) R is an organic radical such as methoxide, ethoxide, propoxide (n- and iso-), butoxide (n-, iso-, sec-, and tert-), amyloxide (n-, sec-, tert-), neopentyloxide or aryloxide,
(c) x corresponds to the valency of the metal M and
(d) n corresponds to the degree of molecular association.

5. Metal complex according to claim 1 characterized by a metal alkoxide to macrocycle molar ratio of 1000:1 to 1:1000, a metal alkoxide to isoprenoid molar ratio of 1000:1 to 1:1000 and a metal alkoxide to linear tetrapyrrole molar ratio of 1000:1 to 1.1000.

6. Metal complex according to claim 1 wherein the macrocycle compound is a phthalocyanine/metal phthalocyanine or a naturally or synthetically occurring porphyrin, preferably chlorophyll or calixarene, preferably 4-tert-butylcalix[4]arene or a cyclodextrin, and/or a natural or synthetically isoprenoid is a carotenoid preferably b-carotene or Curcuma or ginger or squalene and/or a natural or synthetically linear tetrapyrrole is bilirubin and/or an extract containing macrocycles and isoprenoids and linear tetrapyrroles is blue Spirulina or Spirulina or Chlorella.

7. Metal complex according to claim 1 wherein the metal is titanium, zinc, cerium, iron, aluminium, zirconium, silicon, silver, erbium, terbium, barium, gadolinium, yttrium, cobalt, bismuth, calcium, strontium, molybdenum, europium, holmium, boron, sodium, potassium, chromium, manganese, platinum, gold, gallium, tin, lanthanum, copper, tantalum, magnesium, lithium, antimony, indium, vanadium, tungsten or germanium or mixtures thereof.

8. Process for the fabrication of a metal complexes according to claim 1, comprising in any order, simultaneously or sequentially:
(a) mixing (or adding) the at least one metal alkoxide compound (b) with (into) the at least one macrocycle compound and/or at least one isoprenoid compound and/or at least one linear tetrapyrrole (a) in a container (or a substrate such as keratinaceous fiber or other material),
(b) performing mechanochemistry (e.g. by grinding or by milling or by hand macerating the reactants until an intermediate metal complex material (or intermediate tone) is obtained or a finished material is obtained and either
(c) further grinding or milling the intermediate metal complex material to obtain the finished metal complex and optionally letting rest the final material (coloring matter or tone), whereby the powder obtained is a colored material with colors such as blue, red, yellow, orange, violet, pink, green brown or black;
(d) optionally further processing the intermediate metal complex material (or intermediate tone) (e.g. by extrusion, by blending, by bending brake, by hydraulic press, by thermoforming or by injection molding) until the desired material/article is obtained with the desired color.

9. Process according to claim 8 wherein the mechanochemistry includes the use of a mechanical mortar and pestle, high speed milling, ball milling, attrition milling, vortex grinding, planetary milling and extrusion and/or wherein the container is a hand or mechanical mortar and pestle or a hand or comb and a substrate, e.g. proteinous, algal, icroalgal, fungal, bacterial or from yeast material whereby an optional moderate increase of the temperature is applied.

10. Process according to claim 8 further comprising adding at least one additive compound (c) to at least one macrocycle compound and/or at least one isoprenoid and/or at least one linear tetrapyrrole (a) and at least one metal alkoxide (b) simultaneously, or to every reactant (a) or (b), or after the reactants are put into contact or after the final complex is formed, whereby the amount of additive compound is between 0.01 wt % and 50 wt % of the mixtures of compound(s) (a) and compound(s) (b), preferably 5-30 wt %.

11. A method for forming a metal complex or a compound or a material or an article according to claim 1 whereby the method comprises the step of reacting at least one macrocycles and/or at least one isoprenoid and/or at least one linear tetrapyrrole with at least one metal alkoxide by solvent-free mechanochemistry (grinding or milling) whereby the physical form of the colored material both the intermediate metal complex and the finished metal complex, such as powder, presscake, granule, chip or lake, liquid, liquid dispersion, colloids, paste, liquid crystals or flush color is tuned by the conditions of the mechanochemical reaction such as the type of the reactants, the proportion of the reactants, the temperature, the milling/grinding time, the speed, the ball/weight ratio, the milling media, the presence or the absence of additives.

12. Process according to claim 8 comprising
(a) adding the metal alkoxide compound (b) to a phthalocyanine/metal phthalocyanine and/or porphyrin and/or calixarene and/or cyclodextrin and/or b-carotene and/or squalene and/or bilirubin and/or Spirulina and/or Chlorella and/or curcumin compound (a) and/or consecutively or simultaneously adding the additive natural or synthetic monomer/polymer compound (c) in a hand or mechanical mortar whereby the additive compound (c) is selected from
natural or synthetic monomers that participate in condensation or addition polymerizations, e.g. aminoacids (e.g. glycine, tryrosine); peptides; nucleotides; saccharides (e.g. glucose, trehalose or dextrin); isoprene; butadiene; maleic anhydride; vinyl acetate; ethylene; ethylene oxide; vinyl propionate; ethylene glycol; propylene oxide; vinyl acetate; epoxide monomers; styrene; BPA monomer; acrylates (e.g. methyl methacrylate, butyl acrylate, ethyl methacrylate or acrylic acid); vinyl latex or siloxane,
natural or synthetic polymers/copolymers o modifications thereof, e.g., proteins (e.g. collagen, elastin, silkworm/spider silk, refiectin, keratin, collodion, papain); DNA/RNA; carbohydrates/polysaccharide, e.g. cellulose, hemicellulose, regenerated cellulose, cellulose ether/esters, starch, pullulan, glycogen, glucan, pullulan, gum, galactoarabinan, chitin, maltodextrin, chitosan, maltodextrin, carrageenan, albumen; lignin, e.g. from wood, from bark, from delignification process—sulfite pulping or kraft process—; polyvinyl alcohol; polyisoprene, e.g. ruber; polyethylene; polyethylene glycol; polypropylene; unsaturated polyester resins; phenol formaldehyde resins; vinylesters; epoxy resins: polyurethanes: carbon fiber reinforced polymer; polyolefines; polycarbonates; polyacrylates e.g. poly(methyl methacrylate) or butylacrylate; nylon; polystyrene; polysiloxane, e.g. polydimethylsiloxanes, decamethylcyclopentasiloxane or silicone caulk,
(b) grinding or milling the reactants until a homogeneous intermediate and either
(c) further grinding or milling the intermediate metal complex material until a powder material is obtained, whereby the powder obtained is a colored material with colors such as blue, red, yellow, orange, violet, pink, green brown or black; or
(d) further processing the intermediate metal complex material (e.g. by extrusion, by blending, by bending brake, by hydraulic press, by thermoforming or by injection molding) until the desired material/article is obtained with the desired color.

13. Metal complex obtained by the process of claim 8.

14. Composition in the form of an emulsion (water in oil or oil in water), miniemulsion, microemulsion, suspension or dispersion comprising a metal complex according to claim 1 wherein the metal complex is encapsulated in a polymer matrix.

15. Cosmetic, sunscreen, pharmaceutical food, staining, painting, coating, superhydrophobic coating/textile/surface composition comprising a metal complex compound or material according to claim 1.

16. Process for the fabrication of a sunscreen/UV filter/UV absorber product according to claim 1 to prevent premature aging of the keratinous material, to prevent skin or hair disorders, to prevent photodegradation or degradation of a compound/material/surface comprising the following steps:
(a) the intermediate or the finished metal complex material according to any preceding claim is mixed with a vehicle in such a way that the desired sun protection factor is obtained, whereby said vehicle is selected from: a cream base for preparing skin or hair creams such as cetomacrogol creme commercially available, comprising water, decyl oleate, cetaryl alcohol, cetearth-20, sorbitol, sorbic acid, or any cetomacrogol creme; a pasty fatty substance such as waxes, gum or mixtures thereof, an oil, a fatty alcohol, a surfactant, a gel, a powder, a UV filter and/or UV absorber, a skin or hair protector, an emollient, an humectant, an emulsifier, a skin or hair conditioning, a refatting agent, a masking agent, an emulsion stabilizer, a cleansing agent, an antioxidant, an opacifying agent, an aqueous or organic solvent, a viscosity controller, a bulking agent, an abrasive, an anticaking agent, a preservative, a partum, a buffer agent, an antimicrobial, a salt, water or mixtures thereof or any other cosmetically or pharmaceutically acceptable vehicle;
(b) optionally adjusting the desired sun protection factor by the addition of further synthetic or natural filters such as those listed in the EU Cosmetic Ingredient Database.

17. A method of coloring material according to claim 8 which comprises applying to the material being colored, or putting the material being colored into contact with, in any order, successively or simultaneously,
(a) at least one compound selected from macrocycles and/or isoprenoids and/or linear tetrapyrrole
(b) at least one metal alkoxide
(c) and optionally at least one additive, under conditions that the material being colored is absorbed or adsorbed with the compound (a) and left under conditions that no lateral reactions take place or lateral reactions are minimized—e.g. by solvent-free conditions—and then the compound (b), the metal alkoxide, is provided; and by using the hand or the fingers, or a pestle or a comb as pestle tool and the other hand as a container to perform a mechanochemical reaction having the material being colored as substrate in between, wherein the at least one additive (c) can be added to at least one compound (a) or at least one metal alkoxide whereby if an additive such as an aqueous or organic solvent is used to enhance the diffusion of compound (a) or (b), it is preferably let evaporating or drying in the substrate before the next compound is added and if an aqueous or organic herbal/algae/microalgae/fungus/animal compound or material is used, the solvent is let to dry out of the substrate before the next component is added, optionally, if the complex is formed in an external container, it can be applied to the substrate or material being colored and then the method of coloring material of the present claim can be carried out.

18. Use of at least one metal complex (or mixture thereof) either as a dye/pigment itself or in combination with other dyes/pigments or compounds according to claim 1 as a textile dye, keratinous dye, wood/paper dye, food dye, pharmaceutical dye in powder form or as colloids; as catalyst for reactions and polymerizations; as additives in semiconductors, conductors or superconductors; as DNA RNA binding agent; as DNA RNA coating technique or any other coating technique such as superhydrophobic/hydrophobic coating.

19. The mechanochemical synthesis method according to claim 8 wherein the intermediate or the finished metal complex material comprises a metal macrocycle and/or a metal isoprenoid and/or a metal linear tetrapyrrole or a metal macrocycle/isoprenoid/linear tetrapyrrole complex or other combination thereof, either alone or in combination/conjunction with other additives, such as monomers/polymers for the further material functionalization/manufacture/article processing to obtain the finished article.

20. Use of the entire process or part of the process to produce metal macrocycle complexes and/or metal isoprenoid complexes and/or metal linear tetrapyrrole complexes by mechanochemistry (grinding or milling) and/or the metal complexes of macrocycles and/or isoprenoids and/or linear tetrapyrroles obtained thereof according to claim 1 to prepare materials or surfaces or article of manufacture with other properties, such as superamphiphobic or superamphiphilic properties and/or antiviral/antifugal/antibactericide properties.

21. Use of the entire process or part of the process to produce metal macrocycle complexes and/or metal isoprenoid complexes and/or metal linear tetrapyrrole complexes by mechanochemistry (grinding or milling) and/or the metal complexes of macrocycles and/or isoprenoids and/or linear tetrapyrroles obtained thereof according to claim 1 as a step of a selective extraction of biomolecules from natural resources by using aqueous and/or organic solvents.
